(19) United States
(12) Patent Application Publication
(10) Pub. No.: US 2012/0148647 A1
(43) Pub. Date: Jun. 14, 2012

(54) Photoresponsive Sunscreen Composition

(75) Inventors: Bernd Walzel, Zurich (CH); Daniel Fitzgerald, Zurich (CH)

(73) Assignee: Blueshift Pharma GmbH, Zurich (CH)

(21) Appl. No.: 13/391,248

(22) PCT Filed: Aug. 23, 2010
(86) PCT No.: PCT/EP2010/062277

§ 371 Date: Feb. 17, 2012

(30) Foreign Application Priority Data
Aug. 21, 2009 (EP) 09168435.7

(51) Int. Cl. A61K 8/35; A61Q 17/04

(52) U.S. Cl. 424/401; 424/59; 424/60

(57) ABSTRACT

The present invention relates to a composition containing a sunscreen agent capable of undergoing protonation or deprotonation to form a protonated or deprotonated, respectively, sunscreen agent which absorbs, scatters or reflects more or less UV radiation than it does before being protonated or deprotonated, respectively, and a photoresponsive acid or base generating system which is capable of protonating or deprotonating, respectively, said sunscreen agent when the composition is exposed to UV radiation. Further subject matter of the present invention is a microcapsule comprising a sunscreen agent capable of undergoing protonation or deprotonation to form a protonated or deprotonated, respectively, sunscreen agent which absorbs, scatters or reflects more or less UV radiation than said sunscreen agent does before being protonated or deprotonated, respectively, wherein said sunscreen agent is present in said microcapsule substantially in its protonated or deprotonated form that absorbs, scatters or reflects less UV radiation than said sunscreen agent does upon protonation or deprotonation, respectively. The composition and microcapsule according to the invention are particularly useful for providing sunscreen formulations for cosmetic applications and for providing dermatological formulations for medical applications.

The present invention relates to a composition containing a sunscreen agent capable of undergoing protonation or deprotonation to form a protonated or deprotonated, respectively, sunscreen agent which absorbs, scatters or reflects more or less UV radiation than it does before being protonated or deprotonated, respectively, and a photoresponsive acid or base generating system which is capable of protonating or deprotonating, respectively, said sunscreen agent when the composition is exposed to UV radiation. Further subject matter of the present invention is a microcapsule comprising a sunscreen agent capable of undergoing protonation or deprotonation to form a protonated or deprotonated, respectively, sunscreen agent which absorbs, scatters or reflects more or less UV radiation than said sunscreen agent does before being protonated or deprotonated, respectively, wherein said sunscreen agent is present in said microcapsule substantially in its protonated or deprotonated form that absorbs, scatters or reflects less UV radiation than said sunscreen agent does upon protonation or deprotonation, respectively. The composition and microcapsule according to the invention are particularly useful for providing sunscreen formulations for cosmetic applications and for providing dermatological formulations for medical applications.

Solar radiation continuously bombards our planet and encompasses the entire electromagnetic spectrum, including short wavelength high energy cosmic and gamma radiation, longer wavelength lower energy UV radiation, visible light, infrared (IR) radiation, microwaves, and finally, with λ on the order of meters, radio waves. High energy cosmic and gamma rays (λ<10 nm) displace electrons from molecules to form ions, and are thus called “ionizing radiation”. UV, visible, IR, microwave and radio waves lack the energy required for this process, and are therefore classified as non-ionizing radiation.

The UV spectrum is divided based on wavelength into vacuum-UV (λ=10-100 nm), UVC (λ=100-280 nm), UVB (λ=280-320 nm), and UVA (λ=320-400 nm). While the solar spectrum represents a wide range of potential energies and wavelengths, nearly 30-40% of this radiation, including almost all of the most harmful, high energy portions below 295 nm is absorbed in the upper layers of the earth's atmosphere by the ozone layer (Roberts J. Exposure to the sun. In: Auerbach P, ed. Management of Wilderness and Environmental Emergencies. 2nd ed. St. Louis: Mosby, 1989).

Thus, vacuum UV, UVC, and the shortest UVB wavelengths are all blocked by the ozone layer. Conversely, minimal UVA radiation is filtered in the atmosphere before reaching the Earth's surface. The UV radiation that penetrates the ozone layer and reaches the Earth's surface is ca. 10% UVB and 90% UVA at midday (solar noon). UVB intensity is highest at solar noon, and declines thereafter while UVA intensity remains relatively constant throughout the day (Gasparro et al., Photochem Photobiol 1998; 68(3):243-256).

The Global Solar UV Index (UV index) is a measure of the UV radiation level on the Earth's surface and therefore indicates the potential risk for skin damage or disease. The UV index was developed through an international effort by the World Health Organization (WHO) in collaboration with other agencies. It is measured on a scale of 0 (no sun intensity) to around 20 (highest sun intensity), and can be predicted using a computer algorithm, which starts with a UV dose rate at the next “solar noon” (approximately 12:00 p.m.) and incorporates such variables as ozone levels, cloud cover, latitude, elevation, and season. This index is widely publicized on the internet, radio, television, and in newspapers. Since the UV index is influenced by numerous environmental factors it can be very difficult to predict. The UV Index of natural or simulated sunlight can be easily measured using commercially available UV meters or detectors, such as the UV Index Dosimeter (UV888) from Oregon Scientific.

The sun protection factor (SPF) indicates a factor of protection against sunburn. It is commonly defined, for a given sun intensity, as the ratio (Q) of the threshold time of developing erythema (an indication of beginning sunburn) on a skin onto which a sunscreen composition has been applied to the threshold time of developing erythema without application of the sunscreen composition (Pschyrembel/Hunnius Medical and Pharmaceutical Dictionary, Walter de Gruyter 2010). For example, if under a given sun intensity it normally takes one 30 minutes to sunburn, then a sunscreen with SPF 10 will theoretically allow one to stay 10 times longer in the sun (or 300 minutes) before developing sunburn (Department of Health and Human Services FDA, USA. Sunscreen drug products for over the counter use: proposed safety, effectiveness and labelling conditions. Federal Register 1978; 43:38206-69). Twenty years ago most commercially available sunscreens had SPFs of less than 10, but today most manufacturers offer products with SPFs of 15 to 20, and products claiming an SPF of 50 or higher are also available. SPF values may be determined by in-vivo methods as summarised above. Alternatively they may be predicted using in-vitro methodology.

A major limitation common to all known sunscreen formulations is that they provide only static protection against UV radiation and do not address the problem that sun intensity varies greatly with numerous factors including time of day, season, latitude, cloud cover, ozone layer thickness, and altitude. Therefore, the static sun protection afforded by sunscreen agents in practice oscillates between underprotection and overprotection.

This static protection results in two types of performance deficits. At high sun intensity, static sunscreen agents underprotect the user. This is expected to result in the biological effects of increased skin aging, and simultaneously, increased DNA mutation rates, leading to skin cancer. At low sun intensity, static sunscreen agents overprotect the consumer, inhibiting sun tanning. Ideally, sunscreen agents would therefore adapt their protection on the skin to correlate with UV index. Furthermore, and as described above, while the intensity of UVA radiation remains relatively constant throughout the day, the intensity of UVB radiation varies during the day, typically peaking in intensity at solar noon. Analogously therefore, it would be advantageous if sunscreen protection would adapt its protection to correlate with the changing intensity of UVB radiation.

A more recent application in the cosmetics industry is to use sunscreen agents in anti-age and makeup cosmetic products, typically in the range of SPF 8-SPF 15. Consumers typically wear such protection during the day (and even at night), including during the indoors or on rainy days where protection against UV radiation is not desirable (e.g. some exposure of the skin to UV radiation is required for vitamin D3 synthesis, and a slight natural tan is often desired). It would be therefore be advantageous if such anti-age and makeup cosmetic products were available that provided limited or no UV protection at the time of application (for example, SPF 1) and only after exposure to intense sunlight (such as during the period when the individual wearing such a product is exposed to UV radiation during only sunny afternoon of a week) develops a measurable SPF, preferably in the range of SPF 8 to SPF 15.

Another problem faced by all topically-applied sunscreen protection is that once applied, such as to the skin and/or hair, they are subject to progressive physical and chemical removal or degradation. For example, this may occur due to physical abrasion on objects such as clothing, or the sunscreen protection may be at least partially removed or dissolved by moisture, such as by washing, rain, swimming or during other water-based activities. Furthermore, sweat may partially remove or degrade the sunscreen formulation; this problem is even increased if the individual is sweating heavily such as by partaking in rigorous physical activity including sports. Hence, although the protection conferred by a topically-applied sunscreen may be adequate (or overprotect) initially upon its application (such as first thing in the morning), this protection can be reduced progressively during the day by the above factors, including to an extent where at midday there is an inadequate amount of topically-applied sunscreen remaining, and hence providing inadequate protection from UV radiation.

The most common approach to address this problem is simply to improve or otherwise adapt the physical composition and properties of the topical formulation, such as to increase the adhesion or retention of the formulation to the skin, or the resistance of the formulation to removal by abrasion, water or sweat. The skilled person will be aware of the numerous adaptations and improvements made to the composition of topical formulations in order to achieve such effects. However, while this approach may reduce the rate of removal of the formulation; all such formulations are progressively removed, and hence progressively reduce the protection they provide from UV radiation. The only way to maintain (or increase) the level of protection from UV protection with such formulations is therefore to regularly reapply them.

Furthermore, all known commercial organic sunscreens undergo some degree of photodegradation in sunlight. Therefore, sunscreen photostability is of great concern in the sunscreen industry. Sunscreen compositions are typically mixtures of several sunscreen agents, and instability of individual sunscreen agents is responsible for the overall instability of the sunscreen. However, filter combinations of organic and inorganic sunscreen agents are even more prone to photodegradation. For example, irradiation of oxybenzone and titanium dioxide studied by Serpone et al. resulted in about 70% of the oxybenzone being degraded after 20 min of UV exposure (Serpone et al., Photochem Photobiol Sci 2002; 1:970-981). This was a much faster degradation than was observed in a solution containing oxybenzone alone, where 50% was degraded after 260 min. The report by Serpone et al. concluded that oxybenzone degradation was photocatalyzed by titanium dioxide. These two sunscreen agents are present together in many commercially available sunscreen formulations.

Stabilization strategies for photounstable sunscreen agents include adding in the formulation an acceptor that can “quench” the excited state energy of the unstable sunscreen agent. Numerous such stabilizers have been identified for avobenzone, including diethylhexyl 2,6-naphthalate, octocrylene, and methylbenzylidene camphor.

A second strategy has been to remove ingredients known to be deleterious to a particular sunscreen agent's photostability. This includes removing ingredients that are known to otherwise improve overall performance or photostability of the sunscreen.

A third strategy to prevent photodegradation is to alter solvent polarity in the cosmetic formulation, since sunscreen agents are greatly affected by solvent conditions. For example, studies have demonstrated a direct relationship between the dielectric constant of the oil phase and photo decay of various avobenzone-containing filter combinations (Sheath, Sunscreens—Regulations and commercial Development, Third Edition, 2005).

A fourth strategy is to physically isolate the unstable sunscreen agent from ingredients known to be deleterious to its photostability. Encapsulation is a method well studied for this purpose with avobenzone (Schwack et al., GIT Lab J 1997; 1:17-20; U.S. Pat. No. 6,607,713; U.S. Pat. No. 6,468,509).

Ideally, a sunscreen should be 100% photostable, and/or should be resistant to removal or degradation, and herein we describe a novel approach to counteract photodegradation, and/or counteract or compensate for the effects of removal or degradation, of sunscreens through use of a novel photoresponsive sunscreen agent or system that in certain embodiments steadily increases its protection against sunlight over time, bestowing on the sunscreen an overall net photostability and/or the property of counteracting or compensating for the effects of removal or degradation. Furthermore, the instant invention provides a novel photoresponsive sunscreen system that is able to adapt to the intensity of UV radiation, such as the intensity of UVB radiation as it changes through the day. For example, in certain embodiments the protection to UV radiation conferred by the photoresponsive sunscreen system correlates with the intensity of UV radiation to which it is exposed, such as an increase in the protection as the intensity of UV radiation increases.

Rock and Stowell (WO 99/07336) describe photoresponsive sunscreen compositions that include a sunscreen agent that is capable of undergoing an intramolecular photochemical rearrangement, in particular using sunscreen agents that are benzoin derivatives. Recently, Gallardo et al (Photobiol. Sci. 2010, 9: 530-534) describe a similar approach—organic molecules that rearrange under UV light to yield active sunscreen agents—in this case using photoresponsive benzophenones. However, none of the precursor molecules in either of these systems is currently accepted for cosmetic use under current regulations.

It has been shown that a number of organic sunscreen agents can have their UV absorbance spectra modified in the laboratory by changing the pH of non-cosmetic buffers in which they are comprised. For example, changes in pH and different solvent/proton concentrations were shown to affect the protonation form, and hence UV absorbance spectra changes, of 4,4′-diaminostilbene (Zaho et al. J Photochem and Photobiol., 1996, 99: 23-28) and 4-OH-benzophenones (Castro et al.; J Molec. Struc., 2003, 626: 167-178). However, such laboratory experiments do not suggest any solution to provide a photoresponsive sunscreen formulation.

Kaleta et al (U.S. Pat. No. 6,153,176) describes a low pH sunscreen composition that includes 2-phenylbenzimidazole-5-sulfonic acid substantially present in free-acid form (i.e., protonated), optionally together with other organic sunscreen agents. The sunscreen composition so produced is described as still providing adequate sun protection, less likely to be removed by moisture and not burning or stinging the skin/eyes. The sunscreen formulation described by Kaleta et al. is not photoresponsive.

Mullis (WO 88/007222) describes a photochemical system including: (i) a photoacid progenitor compound capable of photoreactive transformation to a photo acid upon exposure to UV radiation; and (ii) a dyestuff capable of undergoing a colour change upon acidification. The system is used to provide a visualisation system (by a colour change) to quantitatively indicate exposure to UV radiation, for example by the use of a bracelet to be worn by an individual made from a material including such a photochemical system. This photochemical system is not a sunscreen system.

Venugopal et al. (Chem. Mater. 1995; 7: 271-276) disclose a system comprising methyl-substituted polyaniline [poly(o-toluidine) (POT)] which changes its UV-absorptive properties upon protonation/deprotonation and a 2-nitrobenzylsulfonate ester as a non-ionic photoacid generator (PAG) which produces sulfonic acid when irradiated with UV light. This system was used in a photolithographic process to produce submicron features in an intrinsically conductive polymer.

Stowell (WO 2007/051198) describes photoresponsive microcapsules, produced from photoactivatable prepolymers, that become porous to a solution within them upon exposure to light. Such microcapsules are used for light-activated control release of their contents that include solvents, fragrances, flavourings, certain cosmetics, herbicides, insecticides, defoliants, fungicides and insect repellents. Amongst possible cosmetics that may be encapsulated within such microcapsules are described certain sunscreen agents. However, this document only disclosed controlled release of such sunscreen agents and not a photoresponsive sunscreen system.

Other photoresponsive microcapsules have been described that: (i) rupture upon irradiation with light due to the generation of gas triggered by such irradiation and a corresponding increase of internal pressure (U.S. Pat. No. 3,301,439; U.S. Pat. No. 4,898,734; Mathiowitz et al 1981, J Applied Polymer Science 26: 809-922); (ii) degrade upon irradiation with light through de-crosslinking of the polymer shell (Yuan et al 2005, Langmuir 21: 9374-9380) or decomposition of the polymer shell (Katagiri et al 2009, Chemistry of Materials 21: 195-197); and (iii) become permeable upon irradiation with light due (Kono et al 1995, J Applied Polymer Science 56: 707-713).

The technical problem underlying the present invention is to provide sunscreen compositions, or components thereof, that overcome the limitations of current sunscreen systems, including the above described limitations, particularly regarding static UV protection and/or the effects of partial removal/degradation or photoinstability.

The solution to the above technical problem is provided by the embodiments of the present invention as defined herein and in the claims.

In particular, the present invention provides a composition comprising
(a) a sunscreen agent capable of undergoing protonation or deprotonation to form a protonated or deprotonated, respectively, sunscreen agent which absorbs, scatters or reflects more or less UV radiation than the sunscreen agent before being protonated or deprotonated, respectively; and
(b) a photoresponsive acid or base generating system which is capable of protonating or deprotonating, respectively, said sunscreen agent when the composition is exposed to UV radiation.

In particular embodiments, the composition of the present invention is a photoresponsive sunscreen composition. The inventive compositions are particularly useful in sunscreen formulations for cosmetic applications or in dermatological formulations for medical applications, preferably such formulations are photoresponsive.

The term “sunscreen agent” according to component (a) of the present invention embraces all molecules capable of absorbing, scattering or reflecting UV radiation for protecting (human or animal) skin or hair against such radiation, and which molecules are capable of undergoing protonation or deprotonation upon which their UV radiation-absorbing, scattering or reflecting characteristics are changed, i.e. the protonated or deprotonated form of the sunscreen agent absorbs, scatters or reflects more or less UV radiation than the sunscreen agent does before it is protonated or deprotonated, respectively.

Therefore, the sunscreen agent of use in the context of the present invention exists in two states, a protonated state (or synonymously, form) and a deprotonated state (form). It is a beneficial aspect of the present invention that at least one of the UV absorbing, scattering and/or reflecting properties of the protonated form of the sunscreen agent compared to the deprotonated form of the sunscreen agent differs to a measurable extent. For example, a sunscreen agent in its deprotonated form may have a comparatively low absorbance of UV light whereas its protonated form absorbs (preferably substantially) more UV light than the deprotonated form. From the foregoing it is clear that other sunscreens useful in the context in the present invention may have a protonated form that absorbs less UV light than the deprotonated form.

Methods to determine if a given sunscreen agent measurably changes its absorbance of UV radiation upon protonation or deprotonation will be readily available to the skilled person, but include a method summarised by the following steps. First, identify a suitable solvent for said sunscreen agent. Such a solvent may be hydrophilic (eg water or glycerine) organic/protic (eg ethanol, isopropanol or octanol), organic/polar aprotic (eg DMSO, acetonitrile or dichloromethane) or organic/unipolar (eg hexane, hexadecane or diethylether). Second, record the UV-Visible (200 nm-800 nm) absorbance spectra of a solution of the sunscreen agent using a suitable photospectrometer (such as a Beckmann DU-640 Spectrometer as described in the examples herein). Third, take an aliquot of the sunscreen UV filter solution and add a strong acid (eg trifluoroacetic acid, or hydrochloric acid, or perchloric acid) and record its UV-Visible absorbance spectra. Neutralize with a base (see next step) to check for reversibility and record the UV-Visible absorbance spectra again. Fourth, take another aliquot of the sunscreen UV filter solution and add a strong base (eg NaOH, KOH, Triethylamine, Tris(hydroxymethyl)aminomethane (TRIS)) and record its UV-Visible absorbance spectra. Neutralize with an acid (see previous step) to check for reversibility and record the resulting UV-Visible absorbance spectra. Fifth, repeat the above steps with (a) more than one acid, (b) more than one base and (c) more than one solvent to exclude artifacts from specific UV-filter-ion interactions, or specific UV-filter-solvent interactions. Conclusion: measureable changes in UV absorbance that are observed in one or more of such repeat experiments for a give sunscreen agent identify that such sunscreen agent is a suitable sunscreen agent for use in the context of the present invention.

As will be appreciated by the skilled person, a sunscreen agent, when used in the context of this invention may be present in equilibrium between both active (with respect to UV radiation absorbing, scattering and/or reflecting) and inactive forms, i.e. between protonated and deprotonated forms of said sunscreen agent. In the “inactive” form, the sunscreen agent shows reduced UV radiation-absorption, scattering or reflecting characteristics compared to the “active” form. The relative amount of these forms will depend on a number of factors, including pKa and concentration of the sunscreen agent, as well as the physico-chemical environment of the sunscreen agent including pH, ionic strength, solvents polarity and the solvents ability to form hydrogen bonds.

From the view of changing the UV absorption/scattering/reflecting properties of the composition, or other applicable aspects of the invention, to a strong extent, it is preferred that the sunscreen agent is present substantially in its respective low or high UV absorbing/scattering/reflecting state (i.e. present substantially in either the protonated or the deprotonated form of the sunscreen agent) before the composition is exposed to UV radiation (or protonated or deprotonated, respectively). “Substantially” in this regard means that most, e.g. at least 80% preferably at least 90%, more preferred at least about 95%, 96%, 97%, 98% or 99% of the sunscreen agent molecules, or even 100% thereof, when used in the present invention are in the stated form, for example in the protonated or deprotonated, respectively, state. As will be appreciated, the actual molar percentage of the stated form of the sunscreen agent present will depend upon a number of factors, particularly including the pH of the environment around the sunscreen agent. With respect to these embodiments, especially when in the composition or microcapsule of the present invention, it is further preferred that this stated, for example protonated or deprotonated, form of the sunscreen agent is the one that absorbs, scatters or reflects less UV radiation than said sunscreen agent does upon exposure of the composition to UV radiation.

If the deprotonated form of the sunscreen agent is the one that absorbs, scatters or reflects more UV radiation than the protonated form, it is preferred that this sunscreen agent is present in the composition according to the invention substantially in its protonated form before the composition is exposed to UV radiation. On the other hand, if the protonated form of the sunscreen agent is the one that absorbs, scatters or reflects more UV radiation than the deprotonated form, it is preferred that this sunscreen agent is present in the composition according to the invention substantially in its deprotonated form before the composition is exposed to UV radiation.

The adaptable sun protecting properties of the composition according to the invention can be expressed or otherwise characterised by the change of Sun Protection Factor (SPF; see above for definition) upon exposure of the composition to UV radiation. Preferred compositions of the present invention show, upon exposure to UV radiation, an increase of about 3, about 4 or about 5 to about 50, preferably about 10, 15, 20, 25, 30, 35 or 40 or more SPF units compared to the composition before exposure to UV radiation. As well as characterising this change in SPF by an absolute amount, the change may also be expressed as a relative change in SPF of the composition according to the invention. In certain embodiments, the SPF of such a composition upon exposure to UV radiation may be at least about 2 times (or fold) to about 25 times, greater than said composition shows before exposure to UV radiation, preferably about 5, 10, 15 or 20 times (or fold) greater. For example, an initial SPF of 2 may increase by 20-fold to show an SPF of about 40. It will be understood however, that such a relative change in SPF cannot exceed the maximum SPF protection currently measureable, i.e. cannot exceed an absolute SPF value of about 45 or 50.

It is further preferred that the sunscreen agent (before exposure of the composition to UV radiation) is substantially present in its protonated or deprotonated form which form absorbs, scatters or reflects less UVA and/or UVB radiation, more preferably absorbs less UVB radiation, than said sunscreen agent does upon exposure of the composition to UV radiation. In certain of such embodiments, said protonated or deprotonated form of the sunscreen agent preferably absorbs less UVB radiation and more UVC radiation than said sunscreen agent does upon exposure of the composition to UV radiation. Such a “blue-shift” effect of the UV absorbance spectra provides a meaningful way for reducing sunscreen protection as UVC radiation of sunlight does not reach the earth's surface. The erythemally effective radiation reaching the earth surface is concentrated in the UVB range with smaller contributions from the UVA range. UVC protection is not relevant to prevent sunburn.

In other preferred embodiments, the sunscreen agent, before exposure of the composition to UV radiation, is substantially present in its protonated or deprotonated form which form absorbs, scatters or reflects less UVA radiation, preferably absorbs less UVA radiation, than said sunscreen agent does upon exposure of the composition to UV radiation.

In the context of the present invention “UV radiation” means light that contains at least part of the UV spectrum, in particular a UVB component (wherein “UV” and “UVB” radiation are as defined above). Typical UV-, especially UVB-containing light is sunlight. In certain embodiments, the UV radiation has a spectrum that is generally equivalent to that of sunlight, for example that of natural sunlight or of simulated sunlight such as provided by sunlight simulators, sunlamps or sunbeds. In preferred embodiments, the UV radiation is comprised in, or is, sunlight, such as natural sunlight or simulated sunlight. The intensity of such UV radiation is sufficient to change at least one of the UV absorbing, scattering and/or reflecting properties of one form of the sunscreen agent compared to the other form of the sunscreen agent to a measurable extent, as may be determined by one or more of the methods described herein. Such an intensity of UV radiation, for example sunlight, may be described as an “effective amount” of UV radiation.

Sunscreen agents for use in the present invention are, e.g. organic sunscreen agents whose absorbance in the UV range is influenced by their protonation state. Other suitable sunscreen agents for use in the present invention are sunscreen agents whose scattering or reflection of light in the UV range is influenced by their protonation state.

The chemical structure of organic sunscreen agents share in common that they are all substituted aromatic compounds whose absorbance in the UV range depends on photochemical excitation of their conjugated p-electron system. Any chemical modifications of the sunscreen agents that affect the conjugated p-electron system will alter the energy difference between the ground and photoexcited state and thereby have a dramatic effect on the absorbance spectrum of the chemical. One such modification is protonation/deprotonation of a sunscreen agent's functional groups.

Organic sunscreen agents that show changes to their UV radiation-absorption, characteristics (for example in the UVA and/or UVB region) upon protonation or deprotonation include sunscreen agents that belong to a class of sunscreen agents selected from the group consisting of: anthranilates, benzophenones, benzotriazoles, camphors, cinnamates, dibenzoyl methanes, imidazoles, malonates, para-aminobenzoic acids, phenols, phenyl triazines, quinones, salicylates and triazones.

In particular embodiments, the sunscreen agent is an organic sunscreen agent selected from the group of those that have been approved for commercial use, for example one approved for use under applicable regulation by the United States Food and Drug Administration (FDA), the European Commission's Scientific Committee on Consumer Products (SCCP) (such as those published by the European Cosmetic Toiletry and Perfumery Association (COLIPA)), the Japanese Ministry of Health, Labour and Welfare (MHW) Medicine Bureau and/or the Australian Therapeutic Goods Administration (TGA). The skilled person will be aware of how to identify whether a particular organic sunscreen agent has been so approved, and currently the following specific organic sunscreen agents (sorted by group) have been so approved:

Anthranilates: menthyl anthranilate. Benzophenones: benzophenone, benzophenone-I, -2, -3, -4, -5, -6, -8, -9, beta 2-glucopyranoxy propyl hydroxy benzophenone, diethylamino hydroxy benzoyl hexyl benzoate. Benzotriazoles: drometrizole, drometrizole trisiloxane, methylene bis-benzotriazolyl tetramethylbutylphenol. Camphors: 3-benzylidene camphor, benzylidene camphor sulfonic acid, camphor benzalkonium methosulfate, 4-methylbenzylidene camphor, poly acrylamido methyl benzylidene camphor, terephthalylidene dicamphor sulfonic acid. Cinnamates: cinoxate, DEA methoxycinnamate, diisopropyl methyl cinnamate, ethylhexyl methoxycinnamate, ferulic acid, glyceryl ethylhexanoate dimethoxycinnamate, isoamyl p-methoxycinnamate, isopentyl trimethoxycinnamate trisiloxane, isopropyl methoxycinnamate, octocrylene. Dibenzoyl methanes: butyl methoxydibenzoylmethane, dimethoxyphenyl-1-(3,4)-4,4-dimethyl-1,3-pentanedione. Imidazoles: disodium phenyl dibenzylimidazole tetrasulfonate, ethylhexyl dimethoxy benzylidene dioxoimidazoline propionate, phenylbenzimidazole sulfonic acid. Malonates: polysilicone-15. Para aminobenzoic acids: ethyl dihydroxypropyl PABA, ethylhexyl dimethyl PABA, glyceryl PABA, PABA, PEG-25 PABA, pentyl dimethyl PABA. Phenols: digalloyl trioleate. Phenyl triazines: bis-ethylhexyloxyphenol ethoxyphenyl triazine.

Salicylates: ethylhexyl salicylate, homosalate, isopropylbenzyl salicylate, salicylic acid, TEA salicylate. Triazones: diethylhexyl butamido triazone, ethylhexyl triazone.

The term “sunscreen agent” according to the present invention also includes, e.g. polymeric substances which change their UV absorption upon protonation or deprotonation. Examples of this type of sunscreen agents useful as component (a) of the present invention include, but are not limited to, polyaniline, poly(N-vinyl pyrrolidone), poly(styrene-N,N-dimethylaminoethyl methacrylate), poly(vinyl pyrrolidone), polyamide, polyindole, polypyrrole, polystyrene sulfonate, polythiophene, polyurea, polyurethane, polyurethane-polyurea, or copolymers containing these polymers as the protonation-sensitive component.

Preferred organic sunscreen agents of use in the present invention are benzophenone derivatives represented by the following formula (I)

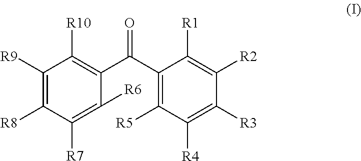

wherein

each of R₁ to R₁₀ is independently selected from the group consisting of hydrogen, hydroxyl, nitro, cyano, amino, halide (in particular F, Cl, Br), straight or branched C₁₋₁₀-alkyl, C₁₋₁₀-alkoxy, C₂₋₁₀-alkenyl, C₂₋₁₀-alkenyloxy, C₆₋₁₀-aryl, optionally substituted with one or more groups selected from hydroxyl, C₁₋₄-alkyl, C₁₋₄-alkoxy, C₂₋₄-alkenyl or C₂₋₄-alkenyloxy, and SO₃M wherein M is hydrogen, a monovalent metal ion or a quarternary ammonium group, preferred examples of this class of organic sunscreen agents are characterised in that at least one of R₁ to R₁₀ is hydroxyl. In alternative embodiments, R₁ to R₁₀ is glucopyranoxy.

Specific examples of such benzophenone derivatives are benzophenone-1, benzophenone-2, benzophenone-3, benzophenone-4, benzophenone-5, benzophenone-6, benzophenone-7, benzophenone-8 and benzophenone-9 whose structures are presented in the following Table

TABLE 1
Names (INCI) and structures of preferred benzophenone derivatives

| | CAS/ | |
|---|---|---|
| INCI Name | EINECS | Structure |
| Benzophenone-1 | | 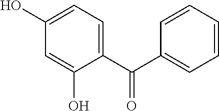 |
| Benzophenone-2 | | 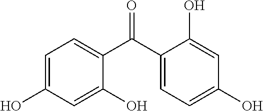 |
| Benzophenone-3 | | 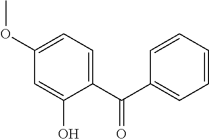 |
| Benzophenone-4 | | 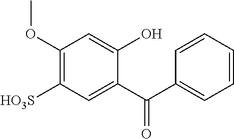 |
| Benzophenone-5 | | 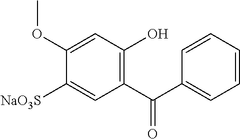 |
| Benzophenone-6 | | 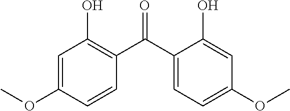 |
| Benzophenone-7 | | 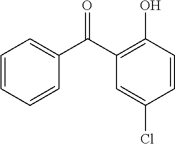 |
| Benzophenone-8 | | 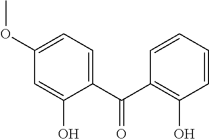 |
| Benzophenone-9 | | 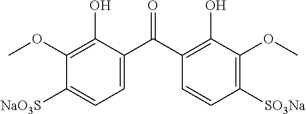 |

In certain preferred embodiments, the sunscreen agent as used in the present invention is one selected from the group consisting of those listed in Table 1 above.

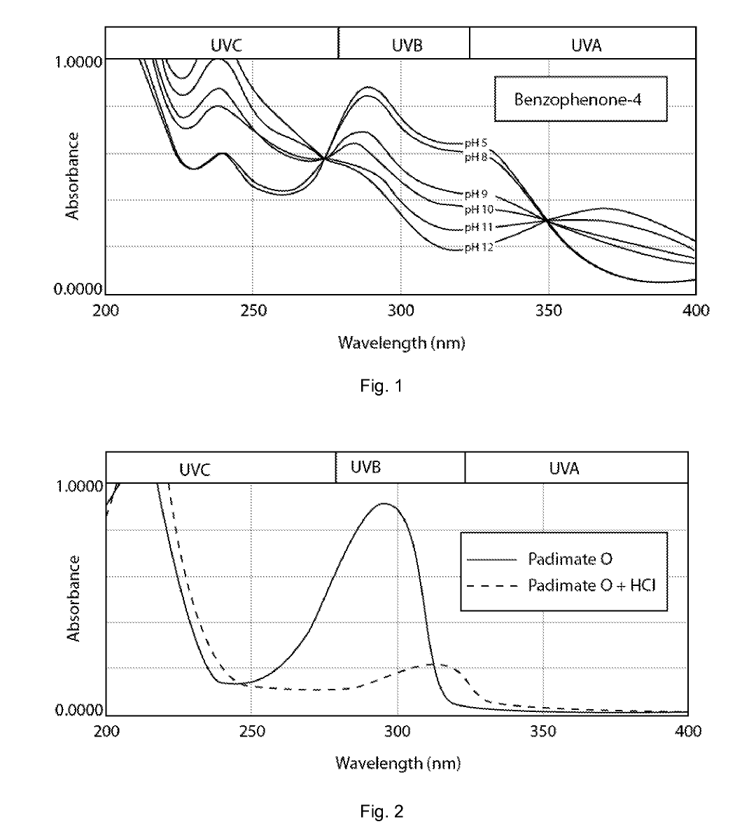

The dependency of absorbance on protonation/deprotonation of benzophenone derivatives in the present invention may be illustrated by using benzophenone-4 as an example. Benzophenone-4 (Table 1) is a water soluble commercial sunscreen agent with very high absorbance in the region of the UV spectrum that causes sunburn (295-320 nm). FIG. 1 shows the effect of pH on the absorption spectrum of Benzophenone-4 in water. Changes in pH from 5 to 11 is expected to convert the protonated molecule of Benzophenone-4 into its deprotonated form. As can be seen in FIG. 1, the critical region of the absorption spectrum for sunburn is greatly affected by the protonation state of Benzophenone-4, with the protonated state being the from that absorbs more UVB radiation. For benzophenone type sunscreen agents it is therefore preferred that they are present substantially in their deprotonated (salt) form in the composition according to the invention before the composition is exposed to UV radiation.

A further preferred class of organic sunscreen agents useful in the context of the present invention are amino phenone compounds of the following formula (II)

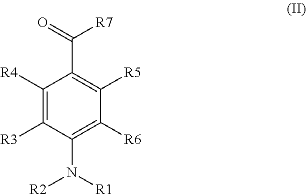

wherein

R₁ and R₂ are each independently selected from H and straight or branched C₁₋₆-alkyl or C₂₋₆-alkenyl, optionally substituted with one or more hydroxyl, R₄ to R₆ are each independently selected from H, hydroxyl, nitro, cyano, amino, halide (F, Cl, Br), straight or branched C₁₋₆-alkyl, C₂₋₆-alkenyl or C₁₋₁₀-alkoxy, optionally substituted with one or more hydroxyl,

R₇ is selected from hydroxyl, straight or branched C₁₋₁₀-alkoxy, C₂₋₁₀-alkenyloxy, C₆₋₁₀-aryl, optionally substituted with one or more groups selected from hydroxyl, C₁₋₆-alkyl, C₁₋₆-alkoxy, C₂₋₆-alkenyl, C₂₋₆-alkenyloxy and C₁₋₁₀-alkoxycarbonyl, or

R₁ is —(CH₂—CH₂O)ₓH, R₂ is —(CH₂—CH₂O)_{y}H, R₇ is —O—(CH₂—CH₂O)_{z}H and each of R₄ to R₆ are H, wherein x+y+z is =25. R₃ may have the same meaning as R₄ to R₆.

From the above definition it is clear that the term “aminophenone” as used herein also embraces the subclass of para-aminobenzoic acids.

It is to be understood that the —NR₁R₂ and —COR₇ substituents may be in ortho, para or meta orientation.

Especially preferred compounds of this class are shown in Table 2.

TABLE 2
Structures of preferred aminophenones

| | CAS/ | |
|---|---|---|
| INCI Name | EINECS | Structure |
| PABA | | 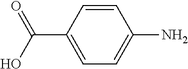 |
| Ethylhexyl Dimethyl PABA (Padimate-O) | | 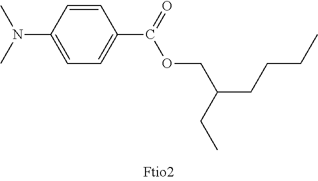 |
| Pentyl Dimethyl PABA (Padimate-A) | | 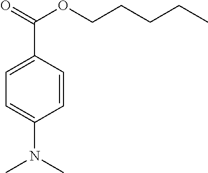 |
| PEG-25 PABA (Lisidimate) | | 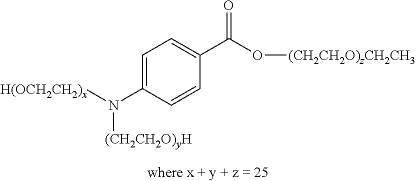 |
| Glyceryl PABA | | 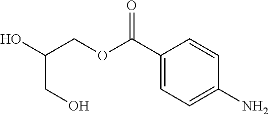 |
| Ethyl Dihydroxypropyl PABA (Roxadimate) | | 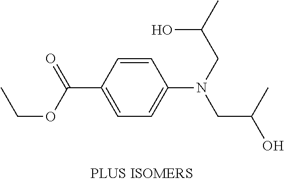 |
| Diethylamino Hydroxy Benzoyl Hexyl Benzoate | | 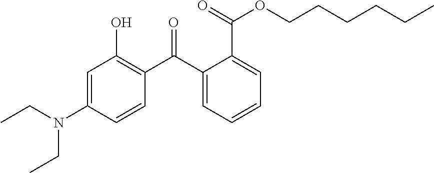 |
| Menthyl Anthranilate (Meradimate) | | 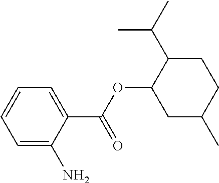 |

In certain preferred embodiments, the sunscreen agent as used in the present invention is one selected from the group consisting of those listed in Table 2 above.

As will be appreciated by the skilled person, the chemical structure of diethylamino hydroxy benzoyl hexyl benzoate (as shown in Table 2 above), has features of both “benzophenone” and “aminophenone” classes of sunscreen agents. From a cosmetic regulatory perspective, this sunscreen agent has been classed as a “benzophenone”. However, in respect of its behaviour to UV absorption upon protonation/deprotonation (i.e. pH sensitivity), diethylamino hydroxy benzoyl hexyl benzoate shows properties analogous to the (aminophenone) PABA-derivatives (i.e., it is largely inactive in its protonated form). Accordingly, and as shown in Table 2, diethylamino hydroxy benzoyl hexyl benzoate may be considered as an aminophenone in the context of this invention.

Padimate-O is an example of exhibiting a dramatic dependency of its absorption between 295 to 320 nm on its protonation state. Padimate-O (Table 2) exhibits a massive difference in absorption between 295-320 nm upon protonation of its amine group (FIG. 2). Protonation of the amine group shifts the absorption maximum of padimate-O from the UVB region to UVC. UVC protection is not relevant to sunburn, since UVC is almost completely absorbed by the ozone layer in the earth's atmosphere, meaning a shift from UVB absorption to UVC absorption decreases the SPF of a sunscreen composition that contains the sunscreen agent Padimate-O (or any other of the aminophenone class). A preferred composition according to the present invention containing aminophenone class sunscreen agent is therefore a composition in which an aminophenone class sunscreen agent is substantially present in its protonated form before the composition is exposed to UV radiation, in particular UVB.

Predicted SPFs can be accurately determined from absorption spectra using standardized calculations based on the erythemal action spectrum, and such predicted SPFs correlate very well with in vivo determined SPFs. Corresponding algorithms for calculating SPF values are known in the art and corresponding computer programs are available; see, for example, http://www.sunscreensimulator.basf.com/Sunscreen_Simulator. Using the absorption spectrum shown in FIG. 2, the predicted SPF of a composition containing the deprotonated form of Padimate-O is approximately 10-fold higher than that of a composition containing the protonated form. With Benzophenone-4 (FIG. 1), the protonated form provides a composition having approximately 3 times the SPF as a composition containing the deprotonated form.

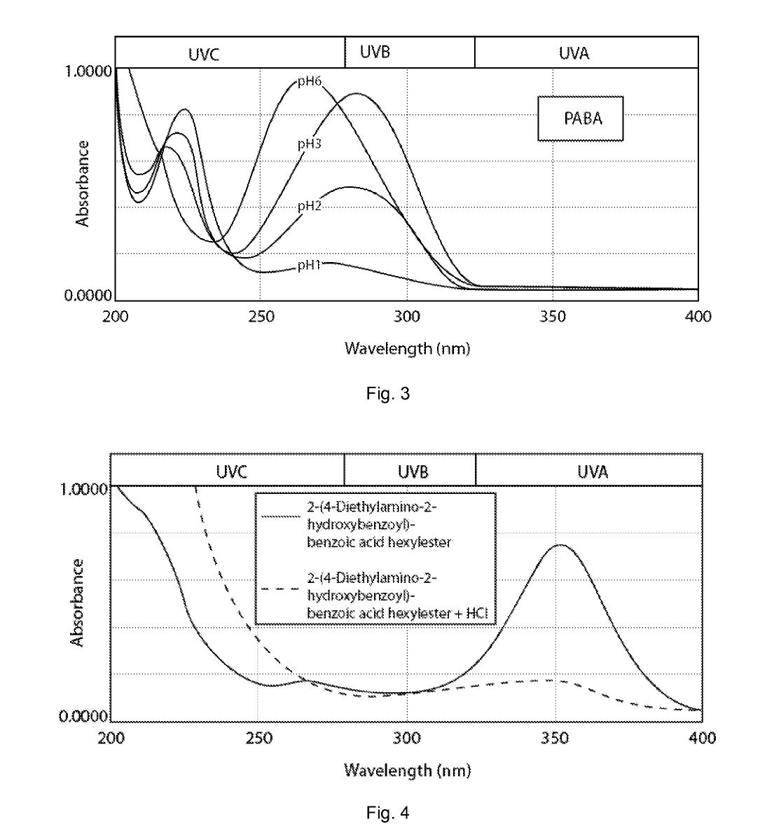

Similar to Benzophenone-4 and Padimate-O, a number of other UVA and UVB sunscreen agents, are also capable of undergoing protonation or deprotonation to form protonated or deprotonated, respectively, sunscreen agents where the protonated or deprotonated, respectively, sunscreen agents absorb either more or less UV radiation than the sunscreen agents before being protonated or deprotonated (see, e.g., FIGS. 3 to 5).

A further preferred class of organic sunscreen agents useful in the context of the present invention is a compound (triazone) of formula (III):

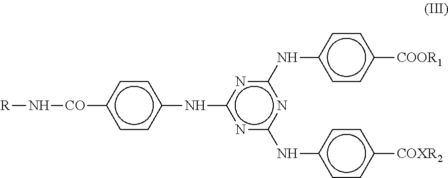

wherein:

R is a straight or branched C₁₋₈-alkyl group, C₅₋₁₂-cycloalkyl, optionally substituted with one or more straight or branched C₁₋₄-alkyl groups; X is an oxygen atom or the group —NH—; R₁ has the same meanings as R, or is hydrogen, a monovalent metal ion, a quaternary ammonium group, or a group of formula (IV)

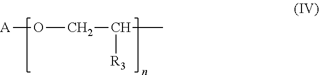

in which A is a straight or branched C₁₋₈-alkyl, C₅₋₁₂-cycloalkyl, or C₆₋₁₀-aryl optionally substituted with one or more straight or branched C₁₋₄-alkyl groups; R₃ is hydrogen or methyl, n is an integer from 1 to 10; R₂ has the same meaning as R when X is —NH—, or has the same meanings as R₁ when X is oxygen.

A further preferred class of organic sunscreen agents useful in the context of the present invention are benzylidene camphor derivatives of formula (V):

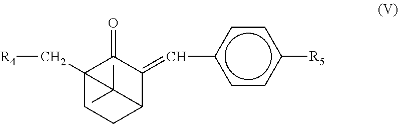

wherein R₄ is hydrogen, or the group SO₃M, in which M is hydrogen, or a monovalent metal ion, or a quaternary ammonium group, and R₅ is hydrogen, methyl, a group SO₃M wherein M is defined as above, or a group of formula (VI) or (VII)

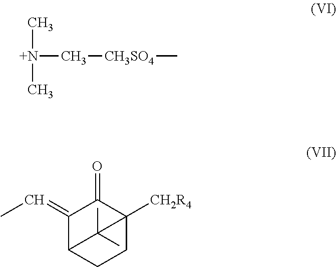

wherein R₄ is as defined above for formula (V). An especially preferred benzyllidene camphor derivative useful in the present invention is camphor benzalkonium methosulfate.

A further preferred class of organic sunscreen agents useful in the context of the present invention are dibenzoylmethane derivatives of formula (VIII):

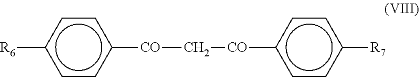

in which R₆ and R₇ are selected independently from hydrogen, straight or branched C₁₋₈-alkyl and straight or branched C₁₋₈-alkoxy.

A further preferred class of organic sunscreen agents useful in the context of the present invention are alkoxycinnamic acid esters of formula (IX)

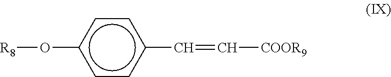

wherein R₈ is a straight or branched C₁₋₈-alkyl group, and R₉ is selected from hydrogen, a straight or branched C₁₋₁₀-alkyl group, a monovalent metal ion and a quaternary ammonium group.

A further preferred class of organic sunscreen agents useful in the context of the present invention are triazinoaniline derivatives of formula (X):

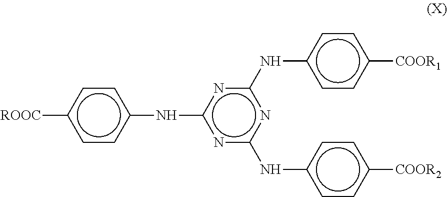

in which R, R₁ and R₂ are defined as above for formula (III).

A further preferred class of organic sunscreen agents useful in the context of the present invention are diphenylcyanoacrylates of formula (XI):

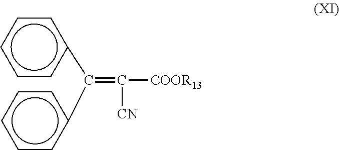

wherein R₁₃ has the same meanings as R₁ defined above for formula (III).

A further preferred class of organic sunscreen agents useful in the context of the present invention are salicylic acid derivatives of formula (XII):

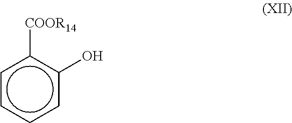

wherein R₁₄ is a straight or branched C₁₋₁₀-alkyl group, a benzyl group optionally substituted with a straight or branched C₁₋₆-alkyl group, a 3,3,5-trimethylcyclohexyl residue, both as a racemate and as any optically active forms, or the group HN⁺(CH₂CH₂OH)₃. In an alternative embodiment, R₁₄ is hydrogen.

A further preferred class of organic sunscreen agents useful in the context of the present invention are benzimidazolesulfonic acid derivatives of formula (XIII):

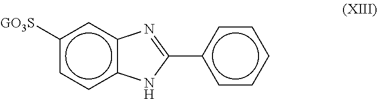

in which G is hydrogen, or a monovalent metal ion, or a quaternary ammonium group.

Yet a further preferred class of organic sunscreen agents useful in the context of the present invention are quarternary ammonium salts of a para-dialkylamino benzamide compound of formula (XIV)

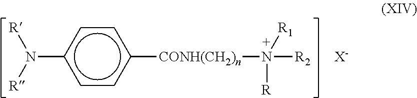

wherein R′ and R″ are each C₁₋₂-alkyl;

n is an integer of from 2 to 6;

R is a linear, branched or cyclic alkyl radical having from 1 to 30 carbon atoms;

R₁ and R₂ are each selected from hydrogen and C₁₋₄-alkyl or, alternatively, R₁ and R₂, together with the attached cationic nitrogen atom can form a 5- to 6-membered heterocyclic ring selected from the group consisting of

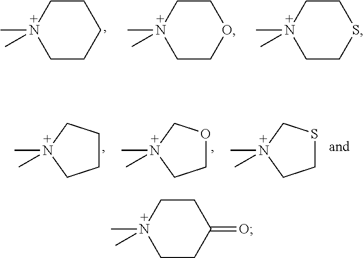

and X is an anion, preferably selected from the group consisting of chloride, bromide, sulphate, sulfonate, haloacetal and aryl sulfonates. A particularly preferred sunscreen agent of this type is dodecyl-[3-(p-dimethylaminobenzamido)propyl]-dimethylammonium tosylate (INCI: Dimethyl PABAmidopropyl Lauryldimonium Tosylate).

In certain embodiments, the sunscreen agent used in the context of the present invention is selected from the group consisting of beta,2-glycopyranoxy propyl hydroxy benzophenone, bis-ethylhexyloxyphenol methoxyphenyl triazine), methylene bis-benzotriazolyl tetramethylbutylphenol, disodium phenyl dibenzimidazole tetrasulfonate, ferulic acid, polysilicone 15, terephthalylidene dicamphor sulfonic acid, and glyceryl ethylhexanoate dimethoxy cinnamate.

In more particular embodiments, the composition of the invention may comprise more than one of the above sunscreen agents, such as a mixture including two or three sunscreen agents described above. For example, the composition may include: (i) a first sunscreen agent that shows a measureable change, upon protonation or deportation, respectively, in the absorbance of UVB radiation, and another sunscreen agent that shows a measureable change, upon protonation or deportation, respectively, in the absorbance of UVA radiation. The sensitivity of their changes in UV absorption upon exposure to UV radiation may differ. For example, the composition may be characterized such that the sunscreen agent having a measurable change in the absorption of UVA radiation is less sensitive to UV exposure than the sunscreen agent having a measurable change in the absorption of UVB radiation. Such a composition would have particular advantages in better correlating with the respective changes in UVA and UVB radiation in sunlight during a day.

The “photoresponsive acid or base generating system” according to the present invention may be realised through photoacid or photobase progenitor compounds (which may as well be called “photoacids” or “photobases”, respectively) which release or accept protons upon exposure to radiation, in particular light, which at least contains a UV component. The change that occurs in these molecules upon absorption of light effectively either releases protons into their chemical environment (photoacids), or accepts protons from their environment (photobases). Photoresponsive acid or base generating systems are capable of reversibly or irreversibly donating protons to or abstracting protons from their chemical environment, i.e. in the context of the present invention donating protons to or abstracting protons from the sunscreen agent, in response to incident radiation.

Some embodiments of photoacid or -base generating systems useful in the present invention may be either
(i) single molecules that liberate or abstract protons in response to incident radiation (monomolecular)
(ii) light triggered chemical reactions involving one or more molecules (e.g. bimolecular) in a chemical reaction that produces (or consumes) protons
(iii) liberation of physically entrapped acid or base

Photoacid progenitors (also called “photoacid generators”) are compounds that can generate acids (Brönsted or Lewis acids) upon irradiation with light. Photoacid generators may be divided into two groups: ionic and non-ionic compounds.

Ionic photoacid generators usually comprise onium salts. E.g. aryldiazonium, diaryliodonium, triarylsulfonium, or triarylphosphonium salts that contain complex metal halides such as BF₄⁻, SbF₆⁻, AsF₆⁻ and PF₆⁻. A variety of onium salts as photoacid generators have since been prepared and the mechanisms for their photolysis have been studied in detail (P. Pappas, J. Imag. Technol. 11, 1466157 (1985)). When onium salts are irradiated at wavelengths in the range of 200-300 nm, they undergo photolysis to form a protic acid. Onium salts have several advantages as photoacid generators. They are thermally stable and may be structurally modified to alter their spectral absorption characteristics.

Non-ionic photoacid progenitor compounds generate an acid from non-ionic compounds upon UV radiation. Examples of acids generated by such compounds include, but are not limited to, carboxylic acids, sulfonic acids, phosphoric acids and hydrogen halides. Non-ionic photoacid generators that generate sulfonic acids upon irradiation include, for example, 2-nitrobenzyl esters of sulfonic acids, imino sulfonate, 1-oxo-2-diazonaphthoquinone-4-sulfonate derivatives, N-hydroxyimide sulfonate, and tri(methanesulfonyloxy)-benzene and its homologues. O-Nitrobenzyl esters of carboxylic acids and 1-oxo-2 diazonaphthoquinone-5-arylsulfonate derivatives generate carboxylic acids upon irradiation. Similarly, triarylphosphate derivatives generate phosphoric acids upon irradiation. Non-ionic photoacid generators benefit from a wide range of solubility in solvents and in polymer films.

Another class of photoacids reversibly change their acidity in the excited state after optical excitation. These photoacids are light-absorbing molecules (chromophores) that are more acidic in the excited electronic state than in the ground state.

Aromatic alcohols such as phenols or naphthols become strong acids when they absorb light (Arnaut et al., J. Photochem. Photobiol. A: Chem. 1993, 75, 1-20). As an example, the pKa of 2-naphthol in the ground state is 9.5, but its pKa in the excited state is 2.8, a change in acidity of around 7 orders of magnitude. Even more dramatic is the change in pKa for 1-naphthol: pKa=9.2 in the ground state and pKa=0.4 in the excited state. Such photoacids usually have a pKa>8 in the ground state and pKa*<2 in the excited state. In certain embodiments, the sensitivity of photoacids to UVA and/or UVB radiation is increased by the presence of photosensitizers. Suitable photosensitizers are described in more detail herein.

Furthermore, persistent and reversible acidification can be achieved with 1-(2-nitroethyl)-2-naphthol (Nunes et al., J. Am. Chem. Soc. 2009, 131, 9456-9462). The process is reversible and can be maintained under continuous irradiation.

Other examples of photoacids are substituted azophenols (Haberfield, J. Am. Chem. SOC. 1987, 109, 6177-6178). After UV light absorption azophenols undergo trans-cis isomerization at the nitrogen-nitrogen double bond. Preferred examples are 2-hydroxy-5-methylazobenzene, 2-hydroxy-3,5,6-trichloro-4′-methylazobenzene, or 2-hydroxy-5,4′-dinitroazobenzene. The cis isomer is more acidic than the trans isomer and thereby such molecules can release a proton upon irradiation into solution. After removal of the UV light source, the molecule returns to the trans isomer and reuptakes the proton.

Another class of photoacid generators are so-called “caged acids” which contain a photolabile “caging” group (Bonetti, et al., C. Chem. Phys. Lett. 1997, 269, 268-273; Barth et al., Biophys. J. 2002, 83, 2864-2871). Photoactivation of such compounds initiates a series of reactions where one of the intermediates is a strong acid that deprotonates and rapidly converts into a photolysis product with low pKa (Laimgruber et al., Angew. Chem., Int. Ed. 2005, 44, 7901-7904).

Nitro-substituted aromatic aldehydes, in particular ortho-nitrobenzaldehyde, are examples of such caged acids (Viappiani et al., Rev. Sci. Instrum. 1998, 69, 270-276). Other useful caged proton compounds include 2-hydroxyphenyl 1-(2-nitrophenyl)ethyl phosphate (caged photosphate) or 1-(2-nitrophenyl)ethyl sulfate (caged sulfate), the latter inducing large pH jumps and can protonate groups that have pKa values as low as 2.2 (Barth et al., Biophysical Journal 83, 2002, 2864-2871).

Another example of photoinduced intermolecular proton transfer has been demonstrated for spirooxazine photochromes (Serena Silvi, et al., J. Am. Chem. Soc. 2007, 129, 13378-13379). These molecules can exist in two isomeric forms, a cyclic spirooxazine form, and an open ring merocyanine structure. Under acidic conditions the protonated merocyanine form is prevalent. Upon irradiation with light the merocyanine is converted to the cyclic spirooxazine form and releases a proton into the solution. This process is reversible and can be repeated many times (Gaeva, et al., Mol. Cryst. Liq. Cryst. 430, 81-88, 2005).

In certain preferred embodiments, the photoacid progenitor compound is one selected from the group consisting of:
a spiroxazine of formula (XV)

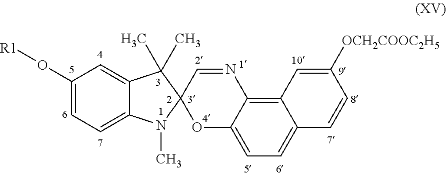

where R₁ is selected from straight, branched or cyclic C₁₋₁₈ alkyl groups. Spiroxazines of this type may be present as single molecules or bound to a polymer; a polymer, such as one selected from chlorinated polymers or copolymers containing poly(vinyl chloride), poly(vinylide chloride);
a naphthol, such as 1-(2-nitroethyl)-2-naphthol (Nunes et al., J. Am. Chem. Soc. 2009, 131, 9456-9462);
an azobenzene such as 2-hydroxy-5-methylazobenzene, 2-hydroxy-3,5,6-trichloro-4′-methylazobenzene, or 2-hydroxy-5,4′-d initroazobenzene; and
a caged acid such as 1-(2-nitrophenyl)ethyl sulphate, or 2-hydroxyphenyl 1-(2-nitrophenyl)ethyl phosphate.

The photochemical generation of a base is a preferred embodiment of a photoresponsive base generating system in the context of the present invention. For example, several structural classes of photolabile compounds generate nitrogen bases upon photochemical decomposition (Suyama et al., Progress in Polymer Science 34, 2009, 194-209). These include, for example, (a) carbamates which generate primary and secondary amines upon photolysis; (b) O-acyloximes which generate primary amines upon photolysis; (c) ammonium salts that generate secondary and tertiary amines or amidines upon photolysis, (d) sulfonamides that generate primary and secondary amines upon photolysis; (e) formamides that generate primary aryl amines upon photolysis; (f) nifedipines that generate hydroxy anions and pyridine derivatives upon photolysis; and (g) α-aminoketones that generate tertiary amines upon photolysis.

The photoresponsive generation of a base according to the present invention can further be realized by heterocyclic compounds which are more basic in their first excited electronic singlet state in comparison to their ground state. Heterocycles of this class include, but are not limited to, acridine and 6-methoxyquinoline (Pines et al., J. Phys. Chem., 1986, 90 (23), 6366-6370; Nachliel et al., J. Am. Chem. Soc. 1987, 109, 1342-1345). Photoexcitation of acridine or 6-methoxyquinoline to their first electronic singlet states is followed by rapid proton abstraction from water, producing hydroxide ions. During the lifetime of the excited state the hydroxide ions are effectively separated and can deprotonate the sunscreen agent component (a) of the composition according to the invention. In certain embodiments, the sensitivity of photobases to UVA and/or UVB radiation is increased by the presence of photosensitizers. Suitable photosensitizers are described in more detail herein

A further class of photoresponsive base generating compounds are carbon bases: The π-electron systems of aromatic alkenes, aromatic alkynes, and aromatic allenes become more basic in their excited state. If carried out in the presence of acids, photoexcitation leads to the overall addition of the acid to a double bond. This type of reaction is particularly fast in moderate to strong acids. The consumption of strong acids leads to decrease of proton concentration (Wan et al., J. Am. Chem. Soc. 1982, 104, 2509-2515). For example, acetic acid is consumed by the photoinitiated addition to the triple bond of diphenylacetylene (Roberts, Chem. Soc., Chem. Commun. 1971,362). Further examples of useful carbon bases in the context of the present invention include, but are not limited to, styrene, 2-vinylnapthalene, 2-naphthylacetylene, nitro-substituted phenylacetylenes, nitro-substituted phenylalkenes, p-nitrophenylacetylene, hydroxy-substituted vinylnapthalenes, hydroxy-substituted naphthylacetylenes and o-hydroxystyrene. A non-exhaustive list of examples of acids useful in combination with carbon bases includes sulphuric acid, acetic acid, trifluorethanol, trifluoracetic acid, hydrochloric acid and perchloric acid.

Yet a further class of compounds useful for photoresponsive generation of bases in the context of the present invention undergoes photodehydroxylation. Thus, methoxy and methoxy and dimethoxy-substituted benzyl alcohols undergo efficient photodissociation releasing hydroxide ions in dilute aqueous acid to form the corresponding benzyl carbocation, which can be trapped by suitable nucleophiles or solvent (Turro et al., Photochem. 1985, 28, 93). The chemical trapping of the intermediate carbocation and the release of hydroxide ions lead to pH increase of the system. Examples of compounds undergoing photodehydroxylation include, but are not limited to, o-hydroxybenzylalcohol, methoxy and dimethoxy-substituted benzyl alcohols, and 2-naphthylmethanol.

Another example of light-induced dehydroxylation has been studied by Irie (J. Am. Chem. Soc., 1983, 105 (7), 2078-2079) who observed a light-induced reversible pH change upon irradiation of aqueous solutions of triphenylmethane leucohydroxide derivatives, such as 4,4′-bis(dimethylamino)triphenylmethane leucohydroxide and sulphonated triphenylmethane leucohydroxide, with UV-light between 280-410 nm. The photochemical reaction efficiently and reversibly leads to the release of hydroxide ions.

Other photodehydroxylations can be generated via photolysis of 9-phenylxanthen-9-ol at 355-337 nm excitation (Minto, J. Am. Chem. Soc. 1989, 111, 8858). Furthermore, steady state irradiation of 9-phenylxanthen-9-ol produced the phenylxanthyl cation and thereby liberated hydroxide ions (Wan et al., J. Org. Chem. 1985, 50, 2881).

Photodehydroxylation of all trans-retinol in nitrogen-saturated acetonitrile by 355-run pulsed laser photolysis gives the all-trans-retinyl cation after liberation of hydroxide ions (Wang et al., J. Photochem. Photobiol. A. Chem 1996, 93, 151). The aromatic alcohol dibenzosuberenol liberates hydroxide ions upon photochemical excitation at 308 nm (Azarani, et al., J. Photochem. Photobiol. A: Chem. 1991, 57, 175, Johnston, et al., J. Phys. Chem. 1989, 93, 7370).

Further compounds undergoing efficient photodehydroxylation comprise di- and triarylmethanol (substituents=methoxy, halogen, nitro, alkyl, sulfone), 9-hydroxyfluorene and its derivatives, retinol and its derivatives, 5-suberol and its derivatives, and pyridoxin (vitamin B6) and its derivatives.

In certain preferred embodiments, the photobase progenitor compound is one selected from the group consisting of, triphenylmethane leucohydroxide (as single molecule or bound to a polymer), 2-Methyl-1-(4-methylthiophenyl)-2-morpholinopropan-1-one (MMMP), 2-Benzyl-2-dimethylamino-1-(4-morpholinophenyl)-butan-1-one (BDMB), trans-retinol, and pyridoxine.

Photochemical reactions of photoacids and photobases can be sensitized with photosensitizers. The presence of photosensitizers typically increases quantum yields of the photochemical reaction. Accordingly, in some embodiments of the invention the photoresponsive acid or base generating system further comprises a photosensitizer Typical photosensitizers include benzophenones, coumarin derivatives, quinines, such as p-benzoquinone, hydroquinone, xanthene dyes, benzoflavine, or setoflavin, 9,10-anthraquinone, benzophenone, 2-chlorothioxanthone, 9-fluorenone and thioxanthone. Other sensitizers include 2-acetonaphthone, ketobiscoumarins and 2-acetonaphthone.

In certain embodiments, the photoresponsive acid or base generating systems according to the invention may be realised in the form of irreversible systems. Alternatively, other embodiments of the photoresponsive acid or base generating systems may be realised in the form of reversible systems.

In a preferred embodiment of the invention, especially with respect to applications on skin and/or hair, the composition is encapsulated. Encapsulation techniques can, for example, be used effectively to diminish or even completely avoid undesired effects of some photoresponsive chemicals of use for protonating/deprotonating sunscreen agents as disclosed herein. A particularly preferred form of encapsulation is the technique of microencapsulation, for example a method that produces microcapsules that comprise a composition of the present invention. Accordingly, in another aspect, the invention relates to a microcapsule comprising a composition, preferably a photoresponsive sunscreen composition, of the present invention.

Microencapsulation is a process of enclosing low-digit to sub-millimetre to micron or nanometer-sized particles of solids or droplets of liquids or gases in an inert shell (or matrix), which in turn isolates and protects them from the external environment, or correspondingly to protect the external environment (such as skin or hair) from the core material. Their inertness is related to the reactivity of the shell with the core material and/or skin or hair.

The microcapsule may, as will be apparent to the skilled person, comprise any suitable material and may be prepared from materials and by methods well known in the art, including as generally described in “Functional Coatings” (Ed. Ghosh, 2006, WILEY-VCH Verlag, Weinheim), “Microencapsulation: Methods and Industrial Applications” 2nd Edition (Ed. Benita, 2006, CRC Press) and “Spray drying handbook” 5th Edition (Ed. Masters, 1994, 1994, Longman Group). Preferably, the microcapsule has a shell or matrix comprising a material selected from the one that belongs to a class of materials selected from the group consisting of: aromatic polymers, diene polymers, epoxy resins, heteroaromatic polymers, heterocyclic polymers, inorganic polymers, phenolic polymers, phenolic resins, poly heterocyclic polymers, poly(α-olefins), polyacetals, polyacrylates, polyalkynes, polyamides, polyaramides, polyesters, polyethers, polyimides, polyisocyanides, polymethacrylates, polyolefines, polysiloxanes, polysulfides, polyureas, polyurethanes, vinyl polymers, vinylidene polymers and perfluoralkoxy polymers.

More preferably the microcapsule has a shell or matrix comprising a material selected from the group consisting of: alkyd resins, bisphenol-A polysulfone, carboxylated ethylene copolymers, Nylon 11, Nylon 12, Nylon 3, Nylon 4,6, Nylon 6, Nylon 6 copolymer, Nylon 6,10, Nylon 6,12, Nylon 6,6, Nylon 6,6 copolymer, Nylon MXD6, silicium dioxide (glass) by sol gel encapsulation, poly(1,3-dioxepane), poly(1,3-dioxolane), poly(1,4-phenylene vinylene), poly(2,6-dimethyl-1A-phenylene oxide), poly(4-hydroxy benzoic acid), poly(4-methyl pentene-1), poly(4-vinyl pyridine), poly(acetylene), poly(acrylamide), poly(acrylic acid), poly(benzimidazol) (PBI), poly(benzobisoxazol) (PBO), poly(benzobisthiazol) (PBT), poly(butadiene) (PBD), poly(butene-1), poly(butyl methacrylate), poly(butylene terephthalate) (PBT), poly(chloral), poly(chloro trifluoro ethylene), poly(chloroprene), poly(cyclohexyl methacrylate), poly(di-n-butyl siloxane), poly(di-n-hexyl siloxane), poly(di-n-hexyl silylene), poly(di-n-pentyl siloxane), poly(di-n-propyl siloxane), poly(diethyl siloxane), poly(dimethyl siloxane), poly(dimethyl silylene), poly(diphenyl siloxane), poly(ether ether ketone), poly(ether imide), poly(ether ketone), poly(ether sulfone), poly(ethyl acrylate), poly(ethyl methacrylate), poly(ethylene-2,6-naphthalate), poly(ethylene), poly(hydridosilsesquioxane), poly(m-phenylene isophthalamide), poly(methacrylic acid), poly(methyl acrylonitrile), poly(methylene oxide), poly(methylphenyl siloxane), poly(methylsilmethylene), poly(methylsilsesquioxane), poly(n-butyl isocyanate), poly(N-methylcyclodisilazane), poly(N-vinyl carbazole), poly(N-vinyl pyrrolidone), poly(p-benzamide), poly(p-chlorostyrene), poly(p-methyl styrene), poly(p-phenylene oxide), poly(p-phenylene sulfide), poly(p-xylylene), poly(phenyl/tolylsiloxane), poly(phenylsilsesquioxane), poly(propyl methacrylate), poly(propylene), poly(pyromellitimide-1.4-diphenyl ether), poly(sulfur nitride), poly(tetrahydrofuran), poly(thiophene), poly(trimethylene oxide), poly(urea), poly(urethane), poly(vinyl acetate), poly(vinyl alcohol), poly(vinyl butyral), poly(α-methylstyrene), poly(∈-caprolactone) and any other material disclosed herein.

Most preferably the microcapsule has a shell or matrix comprising a polymer or co-polymer selected from the group consisting of: poly(acrylonitrile (PAN), poly(carbonate) (PC), poly(chlortrifluor ethylen) (PCTFE), poly(ether sulphone) (PES), poly(ethylene oxide) (PEO), poly(ethylene terephthalate) (PET), poly(ethylene-co-vinyl acetate), poly(ethylene-co-vinyl alcohol), poly(ethylene) high density (HDPE), poly(ethylene) low density (LDPE), poly(methyl methacrylate) (PMMA), poly(methyl trifluoro propyl siloxane), poly(p-phenylene terephthalamide) (Aramide), poly(perfluor ethylen propylen (FEP), poly(perfluoralkoxyl alkan) (PFA), poly(propylene) (PP), poly(styrene-acrylonitrile) (SAN), poly(styrene-co-methyl methacrylate) (SMMA), poly(styrene) (PS), poly(tetrafluor ethylen) (Teflon), poly(vinyl chloride) (PVC), poly(vinyl fluoride) (PVF), poly(vinylidene chloride) (PVDC) and poly(vinylidene fluorid) (PVDF).

The photoresponsive sunscreen composition according to the present invention may also be realized by physically separating the sunscreen agent from the acid or base system where the separation is at least partly removed in a photoresponsive fashion. This aspect is a preferred mode of carrying out the invention, especially with respect to applications on skin and/or hair, since encapsulation techniques can, for example, effectively be used to diminish or even completely avoid undesired effects of some of the chemicals, such as the protonated/deprotonated sunscreen agents, used as disclosed herein.

Accordingly, and in a preferred mode of carrying out the invention, a mode alternative to the photoresponsive acid or base generating system being realised through photoacid or photobase progenitor compounds, the “photoresponsive acid or base generating system” may also be realised through the use of a separation system that “generates” the acid or base for protonation or deprotonation, respectively, of the sunscreen agent by at last partial removal of the physical separation between the sunscreen agent and the acid or base. Before such (partial) removal, the acid or base is not accessible or present in the same environment as the sunscreen agent, and hence upon (partial) removal the (now accessible) acid or base has been “generated” with respect to the sunscreen agent or with respect to the environment, such as a solvent or compartment, that comprises the sunscreen agent. Such embodiments of the photoresponsive acid or base generating system equally may be described as a “photoresponsive acid or base separation system”, or as a “photoresponsive acid or base system”.

Thus, according to an embodiment of the present invention, the photoresponsive acid or base generating system, such as a photoresponsive acid or base (separation) system, respectively, comprises an acid or a base, respectively, present in microcapsules which become at least partially permeable, preferably permeable, for said acid or base, respectively, or for said sunscreen agent upon exposure to UV radiation.

According to a further embodiment of the invention, the photoresponsive acid or base generating system (such as a photoresponsive acid or base (separation) system), i.e. component (b), comprises an acid or a base, respectively, and the sunscreen agent is present in microcapsules which become at least partially permeable, preferably permeable, for said acid or base, respectively, or for said sunscreen agent upon exposure to UV radiation.

Microencapsulation and microcapsules are described above, and such techniques are also applicable to this alternative “separation” mode of the photoresponsive acid or base generating system of the invention.

As mentioned above, photoinduced protonation/deprotonation can be achieved using microencapsulation techniques where the acid (or base) is physically separated from the sunscreen agent. Such separation can be achieved by using a photoresponsive material which is impermeable to acids or bases and thereby prevents direct contact of acid (or base) with the sunscreen agent. In a second step, the photoresponsive material becomes at least partially permeable, preferably permeable, to acids (or bases), such as to protons or hydroxide ions, or is photochemically ruptured upon irradiation with UV-light.

Microcapsule materials useful in the invention include photolabile microcapsules that contain an acid or base. Such microcapsules release the acid or base upon irradiation with light and thereby bring the acid or base into contact with the sunscreen agent which, for example, is present in a formulation external to the microcapsule, or which is generated by a buffered formulation, that is acidic of basic with respect to the sunscreen agent.

Further microcapsule materials according to the invention include photolabile microcapsules that contain the sunscreen agent. Such microcapsules release the sunscreen agent upon irradiation with light and thereby bring the sunscreen agent into contact with the acid or base which, for example, is present in a formulation external to the microcapsule.

Photoresponsive materials, such as those comprising microcapsules may become at least partially permeable by a number of mechanisms. For example, the microcapsule material may become completely and non-selectively permeable by the (at least partial) removal of the separation formed by the material. Such removal can be brought about by photochemical rupture of the microcapsule such that the physical separation is no longer operative.

Photochemical rupture of microcapsules can be achieved by including within the microcapsules a photolabile compound that generates a gas (e.g. nitrogen or carbonoxides) which increases internal pressure that finally ruptures the microcapsule. Corresponding techniques are known in the art, see. U.S. Pat. No. 3,301,439, U.S. Pat. No. 4,898,734 and Mathiowitz et al. (1981) J. Appl. Polymer Sci. 26, 809-822. Further methods to produce gases by UV radiation include, but are not limited to:
photodecomposition of ammonium oxalate (forming gaseous ammonia and gaseous carbon dioxide; non-toxic example); see Nair et al. (1976) J. Phys. Chem. Vol. 80 No. 23, 2552-2555;
UV radiation-induced decomposition of dibenzoyl peroxide (DBP) resulting in the formation of gaseous carbon dioxide, a mechanism known since the 1950s;
UV radiation-induced decomposition of azo-bis-(isobutyronitrilie) (AlBN) resulting in the formation of gaseous nitrogen (see U.S. Pat. No. 3,301,439, U.S. Pat. No. 4,898,734 and Mathiowitz et al., supra);
UV radiation-induced decomposition of ortho-nitrodimethoxyphenylglycine (see Woodrell et al. (1999) Org. Lett. Vol. 1, No. 4, 183-185.

Similar to photochemical rupture of the microcapsules, the (at least partial) removal of the separation can be further achieved by using photolabile capsule materials (e.g. photolabile polymers, photolabile polymer-nanocomposites) that degrade, for example that depolymerize or decompose, under UV radiation, e.g. by photodecrosslinking of o-nitrobenzyl alcohol containing polymers or by photodecrosslinking of polymers containing cinnamate dimers. Such photochemical degradation can completely remove the separation feature, or only remove or degrade portions or parts of the material and hence render the separation component permeable or partially permeable to the acid, base and/or sunscreen agent.

Corresponding photocatalytic degradation, such as decomposition, techniques of capsule shell materials based on a radical mechanism have been recently described by Katagiri et al. (2009) Chem. Mater. Vol. 21, No. 2, 195-197. Sensitized photodecomposition of polymers by UV (UVA/UVB) radiation is known in the art as well (see Torikai et al. (1998) Polymer Degradation and Stability 61, 361-364 (using beta-carotine as photosensitizer) and Torikai et al. (1995) J. Polymer Sci: Part A Polymer Chem. 33, 1867-1871 (using benzophenone as photosensitizer)). Thus, typical photosensitizers of use in this embodiment include carotenes, benzophenones and dibenzoylmethanes.

Further possible mechanisms of degradation employ: (i) the photocleavage of co-polymers containing photolabile monomers within the polymer backbone as break points (see Subramanian (2002) European Polymer Journal 38, 1167-1173); and/or (ii) photodecrosslinking of a crosslinked material such as a polymer or particles (see Yuan, (2005), Langmuir 21, 9374-9380).

Photochemical rupture of microcapsules can be further achieved by including organic polymer-TiO₂ nanocomposites in the microcapsules. TiO₂ acts as photocatalyst to decompose the capsule structure. Photoactive TiO₂ nanoparticles can also be incorporated in the polyelectrolyte shell. TiO₂ nanoparticles adsorbed in a capsule shell act as microheterogeneous photocatalysts performing redox reactions (electron donor/acceptor reactions) with the polymer shell leading to the photodecomposition of the polymer shell.

A further class of photoresponsive separation systems can be achieved by the use of photoresponsive materials, such as co-polymers that become partially permeable to at least the ionic components of the acid, base or sunscreen agent. For example, and as will be appreciated by the skilled person, a microcapsule that upon exposure to UV light becomes permeable (or selectively permeable) to ions, such as hydrogen ions (protons) and/or hydroxide ions will be an effective photoresponsive acid or base generating system. It is sufficient that the separation system, for example the microcapsule or the material comprising the shell or matrix of the microcapsule, becomes permeable to at least the extent that an acid/base equilibrium is established between the prononated or deprotonated sunscreen agent on the one hand and the base or acid, respectively on the other hand. Such equilibrium may be established easily and rapidly by an exchange (by permeation, conduction or transport) of eg protons and/or hydroxide ions from one side of a photoresponsive acid or base separation system to another. Once such equilibrium has been established, the sunscreen agent will shift from the inactive to the active form (or visa versa).

Accordingly, microcapsule systems according to the invention further comprise microcapsules that enclose sunscreen agents within a photoresponsive shell or matrix which is impermeable to acids or bases, including the protons/hydroxide ions of such acids and bases. Upon irradiation with light such photoresponsive shells or matrixes become either permeable to acids (or bases), or become “proton conductor materials” or “hydroxide ion conductor materials” bringing the sunscreen into contact with acid or base, or otherwise establishing acid/base equilibrium between the (protonated or deprotonated) sunscreen agent on the one hand and the base or acid, respectively, on the other. Such materials comprise e.g. copolymers containing leuco dye side groups that liberate hydroxide ions upon photochemical excitation converting the “hydroxide ion isolator” into an “hydroxide ion conductor”. This process is reversible. Upon removal of the light source the polymer reverts back to its isolator state. Specific embodiments of photoinduced ionic conductivity of polymer membranes are disclosed in the art, see, for example, Kubo et al., (1992) Polymers for Advanced Technologies 4, 119-123 (using a co-polymer doped with a spiropyran derivative and LiClO₄); Kubo et al, (1992), Polymer Bulletin 27, 447-450 (using a polymer containing malachite green leuco hydroxide); —Kono et al., (1995) Journal of Applied Polymer Science 56, 707-713 (using a capsule membrane containing triphenylmethane leucohydroxide residues); Berestetsky et al., (2008) J. Phys. Chem. B 2008, 112, 3662-3667 (using poly(4-vinyl pyridine) gel).

Summing up, microcapsules of the invention may become, upon UV radiation, (at least partially) permeable for the acid, base, sunscreen agent, hydrogen ions and/or hydroxide ions and/or rupture, preferably using one or more of the mechanisms as outlined above. A preferred mechanism for rupturing the microcapsule represents the (photoinduced) increase of internal gaseous pressure. In an alternative embodiment, it is preferred to use a photoresponsive material (such as a polymer) that upon UV radiation becomes permeable to (or allows the conduction or transport of) at least hydrogen ions (protons) and/or hydroxide ions. In both these embodiments, the separation component (i.e. the microcapsule shell or matrix) becomes at least partially permeable resulting in acid/base equilibrium to be established between the (protonated or deprotonated) sunscreen agent on the one hand and the base or acid, respectively, on the other.

Further microcapsule systems of the invention comprise microcapsules that have either mononuclear, polynuclear, or matrix morphology. Mononuclear (core-shell) microcapsules contain the shell around the core, while polynuclear capsules have many cores enclosed within the shell. In matrix encapsulation, the core material is distributed homogeneously into the shell material. In addition to these three basic morphologies, microcapsules can also be mononuclear with multiple shells, or they may form clusters of microcapsules. Different morphologies of microcapsules according to the invention are illustrated in FIG. 6.

As described elsewhere herein, and/or within the standard texts cited herein, microcapsules according to the invention may be derived from different microencapsulation processes including extrusion techniques, spray-drying, fluid bed coating, rotating disk, coacervation, solvent evaporation, phase separation, in situ polymerization, interfacial polymerization, miniemulsion, sol gel encapsulation, or layer by layer assembly.

According to certain embodiments of the invention, the term “microcapsules” relates to structures having an average diameter ranging from 1 nm to 200 μm, preferably from 10 nm to 10 μm. However, in other embodiments of the invention, “microcapsules” relate to a structure that has an average largest dimension, preferably a diameter, of between about 15 nm and 1 mm, preferably between about 50 nm and 500 μm, such as about 100, 150, 200, 500, 750 nm, or about 1, 2, 10, 20, 50, 75, 100 or 250 μm.

Bases relevant to the invention in the context of encapsulation techniques include inorganic bases, e.g. sodium hydroxide, potassium hydroxide, magnesium hydroxide, calcium hydroxide, aluminum hydroxide, boric hydroxide, sodium ferulate, magnesium ascorbate, potassium ferulate and calcium carbonate; and organic bases, e.g. primary-, secondary-, tertiary-, or quaternary amines. The bases may be provided in their pure or solubilized form, e.g. in aqueous or organic solvents, in solid or crystalline form, or in mixtures of solid and liquid form.

Suitable bases for use in embodiments of the invention that utilise a separation-based photoresponsive base generating system also include at least one base that belongs to a class of bases selected from the group consisting of: inorganic hydroxides, inorganic oxides, primary- secondary- or tertiary-amines and salts of organic acids. The skilled person will be able to select any number of suitable specific bases from within these groups, which may include one or more of the following specific bases (sorted by group):
Inorganic hydroxides: lithium hydroxide, sodium hydroxide, potassium hydroxide, beryllium hydroxide, magnesium hydroxide, calcium hydroxide, zinc hydroxide, iron hydroxide, aluminium hydroxide.
Inorganic oxides: calcium oxide, magnesium oxide.
Amines: ammonia, ammonium salts (including ammonium chloride, -sulphate, -fluoride, -phosphate, -oxalate, -malate, -acetate, -pyruvate, -citrate, -carbonate), triethanolamine, ethanolamine, aminomethyl propanol, 2-Amino-2-hydroxymethyl-propane-1,3-diol (TRIS), 3-{[tris(hydroxymethyl)methyl]amino}propanesulfonic acid (TAPES), N,N-bis(2-hydroxyethyl)glycine (bicine), N-tris(hydroxymethyl)methylglycine (Tricine), 4-2-hydroxyethyl-1-piperazineethanesulfonic acid (HEPES), 2-{[tris(hydroxymethyl)methyl]amino}ethanesulfonic acid (TES), 3-(N-morpholino)propanesulfonic acid (MOPS), piperazine-N,N′-bis(2-ethanesulfonic acid) (PIPES), 2-(N-morpholino)ethanesulfonic acid (MES).
Salts of organic acids: citrates (including sodium-, potassium-, ammonium-, magnesium-, calcium citrate), acetates (including sodium-, potassium-, ammonium-, magnesium-, calcium acetate), malonates (including sodium-, potassium-, ammonium-, magnesium-, calcium malonate), oxalates (including sodium-, potassium-, ammonium-, magnesium-, calcium oxalate), malate (including sodium-, potassium-, ammonium-, magnesium-, calcium malate), succinate (including sodium-, potassium-, ammonium-, magnesium-, calcium succinate).

In particular embodiments, the base is a base that absorbs UV light, including a sunscreen agent base selected from the group consisting of: cinnamates (including sodium-, potassium-, ammonium-, magnesium-, calcium cinnamates), ferulates (including sodium-, potassium-, ammonium-, magnesium-, calcium ferulates), salicylates (including sodium-, potassium-, ammonium-, magnesium-, calcium salicylates), and benzophenones such as benzophenone-1-2-3-4-5-6-7-8-9 (including sodium-, potassium-, calcium salts thereof).

Acids relevant to the invention in the context of encapsulation techniques include inorganic acids, e.g. perchloric acid, hydrochloric acid, or organic acids, e.g. acetic acid, trifluoroacetic acid, trifluoroethanol, ascorbic acid, ferulic acid, and cinnamic acid.

Suitable acids for use in embodiments of the invention that utilise a separation-based photoresponsive acid generating system also include at least one acid that belongs to a class of acids selected from the group consisting of: inorganic acids, organic acids, hydrochlorides of amines and hydrochlorides of amino acids. The skilled person will be able to select any number of suitable specific acids from within these groups, which may include one or more of the following specific acids (sorted by group):
Inorganic acids: hydrochloric acid, perchloric acid, phosphoric acid, sulfuric acid.
Organic acids: trifluoracetic acid, trichloroacetic acid, trifluoro ethanol, trichloro ethanol.
Hydrochlorides of amines: diethanolamine hydrochloride, triethylamine hydrochloride, 2-amino-2-methyl-1-propanol hydrochloride.
Hydrochlorides of amino acids: glycine hydrochloride, histidine hydrochloride, lysine hydrochloride, tryptophan hydrochloride.

In particular embodiments, the acid is an acid that absorbs UV light, including a sunscreen agent hydrochloride selected from the group consisting of: phenylbenzimidazole-5-sulfonic acid hydrochloride, ethyl dihydroxypropyl PABA (roxadimate) hydrochloride, ethylhexyl dimethyl PABA (padimate O) hydrochloride, glyceryl PABA (lisadimate) hydrochloride, menthyl anthranilate (meradimate) hydrochloride, PABA hydrochloride, PEG-25-PABA hydrochloride, pentyl dimethyl PABA (padimate A) hydrochloride, and diethylamino hydroxybenzoyl hexyl benzoate hydrochloride.

As will be appreciated, sunscreen agents suitable for use in this mode of the invention can be identified or chosen from any of those specified, defined or otherwise described herein. In particular, this mode of the invention may utilise more than one such sunscreen agent.

Photoresponsive acid-base reactions relevant to the invention, in particular in the context of encapsulation techniques, also include the reaction between a protonated form of a first sunscreen agent with a deprotonated form of a second sunscreen agent. In this case the protonated form of a first sunscreen agent transfers a proton to a deprotonated form of a second sunscreen agent. This reaction can be described as an acid/base reaction that established acid/base equilibrium between the (protonated) first sunscreen agent and the (deprotonated) second sunscreen agent.

Thus, according to a preferred embodiment of the present invention component (a) is a first sunscreen agent being in either its protonated or its deprotonated form and component (b) comprises a second sunscreen agent being in its protonated form when said first sunscreen agent is in its deprotonated form and being in its deprotonated form when said first sunscreen agent is in its protonated form such that (b) protonates or deprotonates, respectively, (a). In certain of such embodiments, it is preferred that one sunscreen agent is considered a UVB sunscreen agent, and the other sunscreen agent is considered a UVA sunscreen agent. In another of such embodiments, one sunscreen agent is a sunscreen agent hydrochloride and the other sunscreen agent is a sunscreen agent base, such as those respectively described above.

In one example, a system containing an aminophenone such as padimate-O in its protonated form separated from a benzophenone such as benzophenone-4 in its deprotonated form means that both sunscreen agents are in a low UV absorbance state. By bringing into contact (such as by photoinduced at least partial removal of the separation between these sunscreen agents) the protonated form of the aminophenone like padimate-O with the deprotonated form of the benzophenone, for example, benzophenone-4 a transfer of protons from the aminophenone such as padimate-O to the benzophenone such as benzophenone-4 is initiated (eg, starting an acid/base reaction to establish acid/base equilibrium between the aminophenone and benzophenone sunscreen agents) converting the low absorbance form of the aminophenone (like padimate-O) into its high absorbance form and in parallel converting the low absorbance form of the benzophenone (e.g. benzophenone-4) in its high absorbance form. This type of reaction would not necessarily require additional photoacids or photobases. Hence, such first and second sunscreen agent systems are preferred embodiments of the invention, especially with respect to applications on skin and/or hair, as they avoid possible undesired effects of such photoresponsive chemicals.

In certain embodiments, the photoresponsive acid or base generating systems according to the invention using a photoresponsive separation system such as microcapsules may be realised in the form of irreversible systems. Alternatively, other embodiments of the photoresponsive acid or base generating systems according to this mode of the invention may be realised in the form of reversible systems, e.g. by using reversible photoacid or photobase progenitor compounds as disclosed herein.

Microcapsules may be formed from the materials or by the processes described elsewhere herein. In particular, polymer materials for forming microcapsules may contain polymers or copolymers of acrylic esters, acrylics, acrylonitrile, butadiene, carboxylated styrene-butadiene rubber, chloroprene and copolymers, natural rubber, poly (3,4-ethylenedioxythiophene), poly(diallyl dimethyl ammonium chloride), poly(ethylene glycol), poly(ethylene oxide), poly(N-vinyl pyrrolidone), poly(phenylene vinylene), poly(styrene-N,N-dimethylaminoethyl methacrylate), poly(vinyl alcohol), poly(vinyl alcohol-co-acetate), poly(vinyl methyl ether), poly(vinyl pyrrolidone), Polyacetylene, poly(acrylic acid), polyacrylics, polyalkyl cyanoacrylates, polyamide, polyaniline, polyester, polyindole, Polymer materials may contain polymers or copolymers of polymer/silica nanocomposite particles, polymethyl methacrylate, polypyrrole, polystyrene sulfonate, polystyrene, polystyrene/silica nanocomposite particles, polythiophene, polyurea, polyurethane, polyurethane-polyurea, polyvinyl acetate, pure acrylics, styrene/acrylate copolymers, styrene/acrylic copolymers, styrene-butadiene rubber, vinyl acetate, vinyl acetate/butyl acrylate copolymers, vinyl acetate/dibutyl maleate copolymers.

Encapsulation may also be achieved by sol-gel encapsulation.

The microcapsules may contain fillers or pigments such as metal oxides, (e.g. titanium dioxide (anatase), titanium dioxide (rutile), alumina, zinc oxide, iron oxide, silica); or aluminosilicates (e.g. kaolin, montmorillonite, or laponite); or insoluble salts (e.g. calcium carbonate, barium sulfate).

From the foregoing, it is evident that the present invention is also directed to microcapsules as disclosed herein as such. Thus, in another aspect the present invention provides a microcapsule comprising a sunscreen agent capable of undergoing protonation or deprotonation to form a protonated or deprotonated, respectively, sunscreen agent which absorbs, scatters or reflects more or less UV radiation than said sunscreen agent does before being protonated or deprotonated, respectively, wherein said sunscreen agent is present in said microcapsule substantially in its protonated or deprotonated form that absorbs, scatters or reflects less UV radiation than said sunscreen agent does upon protonation or deprotonation, respectively. Preferably the sunscreen is an organic sunscreen agent which absorbs more UV radiation before being protonated or deprotonated, respectively, and/or is present in said microcapsule substantially in its protonated or deprotonated form, respectively that absorbs less UV radiation.

Preferred embodiments of the microcapsule have already been elaborated before, but especially preferred microcapsules according to the invention contain the sunscreen agent which is present substantially in its protonated form before the microcapsule is exposed to UV radiation. In other embodiments, the sunscreen agent is present substantially in its deprotonated form before the microcapsule is exposed to UV radiation. Preferred sunscreens for use in the microcapsules are specified, described or defined herein. In the context of providing a “functional” microcapsule (which may also be described as a photoresponse microcapsule) for protonating/deprotonating sunscreen agents as described above upon exposure to UV radiation (i.e. to provide a photoresponsive sunscreen system in the sense of the present invention) the microcapsule according to the invention preferably
becomes at least partially permeable for said sunscreen agent or for an acid or a base, respectively, upon exposure to UV radiation; or
further comprises a photoacid or photobase progenitor compound, preferably as defined before, respectively;
   wherein the meaning of, or particular embodiments of these modes, can readily be identified or selected from those specified, described or defined above.

It is to be understood that compositions or microcapsules according to the present invention may contain more than one sunscreen component (a) and more than one photoresponsive acid or base generator (b).

In another aspect, the invention relates to the use of a photoresponsive acid or base generating system to protonate or deprotonate, respectively, a sunscreen agent in a cosmetic sunscreen formulation, a dermatological formulation or a microcapsule, upon exposure of said formulation or microcapsule to UV radiation wherein said sunscreen agent absorbs, scatters or reflects more or less UV radiation than said sunscreen agent does upon protonation or deprotonation, respectively.

In other aspects, the invention relates to the use of a photoresponsive acid or base generating system in the preparation, such in manufacture, of a photoresponsive sunscreen composition or photoresponsive microcapsule, or in the preparation of a cosmetic sunscreen formulation or a dermatological formulation, preferably in the manufacture of a photoresponsive such formulation. In certain embodiments of this aspect, the photoresponsive acid or base generating system is one selected from one specified or described herein, and/or such composition, microcapsule or formulation is one selected from one specified or described herein.

With regard to these inventive uses, the meaning of, or particular embodiments of, features described therein can readily be identified or selected from those specified, described or defined above.

Further subject matter of the present invention relates to a method for the preparation of a composition as defined above comprising the step of combining or admixing components (a) and (b). Said combining or admixing may include mixing, emulsifying and/or encapsulation. In certain embodiments, such encapsulation may form a microcapsule comprising the components (a) and (b).

In certain embodiments of this method, the sunscreen agent, such as one described, specified or defined herein, is protonated or deprotonated, respectively, to provide the protonated or deprotonated form of the sunscreen agent that absorbs, scatters or reflects less UV radiation than prior to said protonation or deprotonation. Accordingly, in certain embodiments, the method further comprises a step of protonating or deprotonating said sunscreen agent, where such protonation/deprotonation step may occur before or after the step of combining or admixing components (a) and (b).

In other embodiments of this method, components (a) and (b) are combined or admixed together with a cosmetically or dermatologically acceptable carrier. Preferably, the cosmetically or dermatologically acceptable carrier is buffered to a pH of between about 4.0 to about 9.0, such as a pH of about 4.5, 5.0, 5.5, 6.0, 6.5, 7.0, 7.5, 8.0 or 8.5.

Another aspect of the invention relates to a method for the preparation of a microcapsule of the invention, said method comprising the step of encapsulating a sunscreen agent to form a microcapsule, wherein said sunscreen agent is capable of undergoing protonation or deprotonation to form a protonated or deprotonated, respectively, sunscreen agent which absorbs, scatters or reflects more or less UV radiation than said sunscreen agent does before being protonated or deprotonated, respectively. In certain embodiments, said sunscreen agent is present in said microcapsule substantially in its protonated or deprotonated form that absorbs, scatters or reflects less UV radiation than said sunscreen agent does upon protonation or deprotonation, respectively. In other certain embodiments said method further comprises the step of protonating or deprotonating, said sunscreen agent to provide the protonated or deprotonated form of said sunscreen agent that absorbs, scatters or reflects less UV radiation than prior to said protonation or deprotonation. Such protonation (or deprotonation) step may occur before or after the encapsulation step. In certain embodiments, the sunscreen agent is an organic sunscreen agent selected from those described, specified or described herein. Preferably, such method produces a microcapsule comprising such a sunscreen agent that is present substantially in the protonated or deprotonated form of said sunscreen agent that absorbs, scatters or reflects less UV radiation than prior to said protonation or deprotonation.

In the applicable aspects of the invention, the steps for encapsulation to form microcapsules may be conducted according to any number of general methods known to the skilled person, including those analogous to the methods described herein.

In another aspect, the invention relates to a method for the preparation of a cosmetic sunscreen formulation, said method comprising the step of combining or admixing the composition of the invention with a cosmetically acceptable carrier. The invention also relates to a method for the preparation of a dermatological formulation, said method comprising the step of combining or admixing the composition of the invention with a dermatological acceptable carrier. In certain embodiments of these aspects, the composition of the invention is included in a microcapsule, such as a microcapsule described herein or prepared as described herein.

The inventive combination of a photoresponsive acid or base generating system with a sunscreen agent whose UV absorbance (or UV reflectance or UV scattering) is dependent on its protonation state therefore results in a photoresponsive sunscreen system. The photoresponsive sunscreen systems of the present invention have at least three applications in cosmetics or medicine:
Reversible protonation/deprotonation of sunscreen agents by sun-activated acid or base generating systems allow sunscreen agents to adapt their UV protection live on the skin to correlate with the UV index of incoming sunlight.
Irreversible protonation/deprotonation of sunscreen agents by sun-activated acid or base generating systems allow sunscreen agents to counteract the photodegradation common to all sunscreens. This can be accomplished through light-dependent, irreversible conversion of a first sunscreen agent into a second sunscreen agent through protonation/deprotonation, where the second sunscreen agent displays a greater protection against UV light than the first sunscreen agent. This steady increase in protection by one sunscreen agent in a formulation counteracts photodegradation of other sunscreen agents and thereby bestows on the sunscreen an overall net photostability.
Irreversible protonation/deprotonation to protect from sun overexposure. A sun-dependent reaction with an irreversible photoacid/photobase acts as a dosimeter to gauge received UV dose. After having received a defined dose of UV-radiation that ensures tanning, a reaction is triggered at a given threshold of reacted photoacid/photobase to completely block UV light. Thus starting with e.g. SPF 1, the composition reaches a determined threshold of UV dose and then suddenly and abruptly changes to e.g. SPF 20. An analogous effect could be achieved in the UVA region with a jump in protection as measured by the Boots Star system. Especially this aspect of the present invention can be realized by using microencapsulation techniques as described above in which microcapsules are provided that break or become permeable after receiving a given UV dose and release an acid or base.

In another application, the photoresponsive sunscreen systems of the present invention may be used in cosmetics or medicine to:
Counteract or compensate for the (at least partial) removal and/or degradation common to all topical sunscreen formulations. This can be accomplished through light-dependent, conversion of a first form of a sunscreen agent into a second form of the sunscreen agent through protonation/deprotonation, where the second form of the sunscreen agent displays a greater protection against UV light than the first form of the sunscreen agent. This steady increase in protection by one sunscreen agent in a formulation counteracts (partial) removal and/or degradation of the formulation and thereby bestows on the sunscreen an overall net stability on the photoprotective properties of a topical sunscreen formulation.

As will be readily appreciated by the skilled person upon the disclosure of the present invention, the particular constituents of a composition, microcapsule or formulations of the invention may vary depending on its desired application or properties. For example, by varying the amounts, identity and concentrations of the compounds such as the sunscreen agent, acid/base or photoresponsive acid or base generating system, the characteristics or properties of the composition, microcapsule or formulation may be varied as desired. In particular, the material used or dimension/thickness of microcapsules of the invention may vary as required in order to achieve a particular property, such as the sensitivity of the photoresponsive sunscreen system to UV radiation.

The present invention is therefore further directed to a cosmetic sunscreen formulation containing the inventive composition or the “functional” microcapsules as defined above together with a cosmetically acceptable carrier. Also, the present invention is directed to a medical (in particular, dermatological) formulation containing the composition as defined herein or the “functional” microcapsules as defined above together with a dermatologically acceptable carrier. Such formulations may be described as “photoresponsive”.

According to the present invention the terms “cosmetically acceptable carrier” and “dermatologically acceptable carrier” are used interchangeably and relate to a corresponding base composition comprising conventional cosmetic and/or pharmaceutical vehicles and/or diluents and/or adjuvants and/or additives and/or additional active ingredients.

The composition or microcapsule of the invention for use in medicine, or the dermatological formulation comprising the same, may not differ in physical composition from the composition or microcapsule of the invention when used for non-medical purposes or from the cosmetic sunscreen formulation. However, in certain embodiments the composition or microcapsule of the invention for use in medicine, or the dermatological formulation, may include additional active ingredients. For example, such compositions, microcapsules or formulations may include pharmaceutically active ingredients such as soothing agents, analgesics, moisturizers, anti-inflammatory agents, anti-infective agents, wound-healing agents and/or anti-cancer agents such as anti-melanoma agents.

The composition or microcapsule, or the sunscreen formulation (such as the cosmetic and/or the dermatological formulation) according to the invention may comprise (in addition to the sunscreen agent capable of being protonated/deprotonated as defined above) one or more further sunscreen agent(s) which may be selected from any known organic or inorganic sunscreen agents.

Organic sunscreens in this context include, but are not limited to, 3-benzylidene camphor, benzylidene camphor sulfonic acid, β-2-glucopyranoxy propyl hydroxyl benzophenone, bis-ethylhexyloxyphenol methoxyphenyl triazine, butyl methoxydeibenzoylmethane, camphor benzalkonium methosulfate, cinoxyte, DEA methoxycinnamate, diethylhexyl butamido trazone, digalloyl trioleate, diisopropyl methyl cinnamate, dimethoxyphenyl-[I-(3,4)J-4,4-dimethyl 3-pentanedione, disodium phenyl dibenzylimidazole tetrasulfonate, drometrizole, drometrizole trisiloxane, ethylhexyl dimethoxy benzylidene dioxoimidazoline propionate, ethylhexyl methoxycinnamate, ethylhexyl salicylate, ethylhexyl triazone, ferulic acid, glyceryl ethylhexanoate dimethoxycinnamate, homosalate, isoamyl p-methoxycinnamate, isopenfyl trimethoxycinnamate trisiloxane, isopropylbezyl salicylate, isopropyl methoxycinnamate, menthyl anthranilate, 4-menthylbenzylidene camphor, methylene bis-benzotriazolyl tetramethylbutylphenol, octocrylene, phenylbenzimidazole sulfonic acid, polyacrylamido methylbenzylidene camphor, polysiliconce-15, salicylic acid, TEA salicylate and terephthalylidene dicamphor sulfonic acid, gallic acid, protocatechuic acid, gentisic acid, p-hydroxybenzoic acid, vanillic acid, isovanillic acid, syringic acid, caffeic acid, p-coumaric acid, ferulic acid and sinapinic acid.

In particular embodiments, the at least one further organic sunscreen agent is selected from any organic sunscreen agent that is disclosed herein, and preferably is one that has been approved for commercial use, for example approved for use under applicable regulation by the United States Food and Drug Administration (FDA), the European Commission's Scientific Committee on Consumer Products (SCCP) (such as those published by the European Cosmetic Toiletry and Perfumery Association (COLIPA)), the Japanese Ministry of Health, Labour and Welfare (MHW) Medicine Bureau and/or the Australian Therapeutic Goods Administration (TGA).

In certain embodiments the at least one further organic sunscreen agent is a combination of at least two sunscreen agents: a UVA sunscreen agent and a UVB sunscreen agent. The skilled person will readily appreciate whether an organic sunscreen agent is a “UVA” or a “UVB” sunscreen agent. In other certain embodiments, the at least one further organic sunscreen agent is one that shows limited sensitivity to changes in pH in regards to UV absorption, including the sunscreen agents butyl methoxydibenzoylmethane, ethylhexyl methoxycinnamate or cinoxate.

Additional inorganic sunscreen agents may be selected from metal oxides having an atomic number ranging from 10 to 40, preferably titanium dioxide and zinc oxide. Such metal oxides may be present at a concentration of between about 1% and 30% (w/w), most preferably at a concentration of about 5%, 10%, 15%, 20% or 25% (w/w).

In certain embodiments, the composition, microcapsule, or (cosmetic or dermatological) formulation of the invention includes both an inorganic sunscreen agent and at least one further organic sunscreen agent (in each case as specified or defined above), preferably a combination of at least two sunscreen agents: a UVA sunscreen agent and a UVB sunscreen agent.

In certain preferred embodiments, the inorganic sunscreen agent and/or the at least one further organic sunscreen agent is present in the composition, microcapsule or formulation in an amount to provide a composition or formulation that before exposure to UV radiation shows an SPF of between 1 and 45, preferably an SPF of about 2, 5, 10, 15, 20, 25, 30, 35 or 40.

The cosmetic sunscreen formulation of the present invention, or the dermatological formulation of the invention, may be in the form of a suspension or dispersion in solvents or fatty substances, or alternatively in the form of an emulsion or micro emulsion (in particular, of O/W or W/O type, O/W/O or W/O/W-type, wherein O stands for oil phase and W stands for water phase), such as a cream, a paste, a lotion, a thickened oil or a milk, a vesicular dispersion in the form of an ointment, a gel, a solid tube stick or an aerosol mousse, may be provided in the form of a mousse, for a foam or spray foams, sprays, sticks or aerosols or vibes. Examples of cosmetic or dermatological preparations are skin care preparations, in particular, body oils, body lotions, body gels, treatment creams, skin protection ointments, moisturizing gels, moisturizing sprays, revitalizing body sprays or lip stick formulations.

Cosmetic compositions, such as a cosmetic sunscreen formulation, or dermatological formulations for use in the present invention may further comprise usual cosmetic or dermatological adjuvants and/or additives such as preservatives/antioxidants, fatty substances/oils, water, organic solvents, silicones, thickeners, softeners, emulsifiers, additional light screening agents, antifoaming agents, moisturizers, frequenters, surfactants, fillers, sequestering agents, anionic, cationic, non-ionic or amphoteric polymers or mixtures thereof, propellants, acidifying or basifying agents, dyes, colorants, pigments or nanopigments, light stabilizers, insect propellants, skin tanning agents, skin whitening agents, antibacterial agents, preservative active ingredients or any other ingredients usually formulated into cosmetic or dermatological preparations.

The necessary amount of the cosmetic/dermatological adjuvants, additives and/or additional active ingredients can, based on the desired end product, easily be chosen by a person skilled in the art.

In preferred modes of practicing the invention, the cosmetic or dermatologic adjuvants, additives and/or additional active ingredients comprise a buffered environment, preferably an environment buffered to a pH of between about 4.0 to about 9.0, such as a pH of about 4.5, 5.0, 5.5, 6.0, 6.5, 7.0, 7.5, 8.0 or 8.5.

The present invention further relates to a cosmetic method for protecting human skin and/or hair against UV radiation comprising the step of applying an effective amount of the cosmetic sunscreen formulation onto (usually human or animal) skin and/or hair.

The photoresponsive sunscreen composition of the invention and the “functional” microcapsules as defined above are for use in medicine (for example for the manufacture of pharmaceutical, in particular dermatological preparations), especially for the prevention of skin cancer or sunburn. The invention is therefore also directed to a medical method comprising the application of an effective amount of the dermatological formulation onto (preferably human or animal such as pig) skin and/or hair.

From the foregoing description it is also evident that the present invention provides a method for modifying the SPF or the Boots star rating of a cosmetic or dermatological formulation as defined above comprising the step of exposing said formulation to an effective amount of UV radiation. Furthermore, in another aspect, the invention relates to a method for the protonation or deprotonation of a sunscreen agent comprised in the composition or microcapsule of the invention, or in the cosmetic or dermatological formulation of the invention, said method comprising the step of exposing the foregoing, respectively, to UV radiation, preferably to an effective amount of UV radiation. In certain embodiments, such protonation or deprotonation causes an increase or decrease in the absorption, scattering or reflection of UV radiation by said sunscreen agent, preferably an increase, and most preferably an increase in the absorption of UV radiation.

The Figures show:

FIG. 1 shows a graphical representation of absorbance spectra (wave lengths from 200 to 400 nm; absorbance in arbitrary units) of benzphenone-4 at different pH values (pH 5, pH 8, pH 9, pH 10, pH 11, pH 12) in ethanol/water solution. The pH was adjusted with NaOH/HCl in aqueous solution.

FIG. 2 shows a graphical representation of absorbance spectra (wave lengths from 200 to 400 nm; absorbance in arbitrary units) of padimate-O in acetonitrile before and after addition of hydrocholoric acid (HCl) in an equimolar ratio of padimate-O/HCl.

FIG. 3 shows a graphical representation of absorbance spectra (wave lengths from 200 to 400 nm; absorbance in arbitrary units) of PABA at different pH values (pH 1, pH 2 pH 3, pH 6) in aqueous solution. The pH was adjusted with NaOH/HCl in aqueous solution.

FIG. 4 shows a graphical representation of absorbance spectra (wave lengths from 200 to 400 nm; absorbance in arbitrary units) of 2-(4-diethylamino-2-hydroxybenzoyl)-benzoic acid hexylester in acetonitrile before and after addition of hydrocholoric acid (HCl) in an equimolar ratio of 2-(4-diethylamino-2-hydroxybenzoyl)-benzoic acid hexylester/HCl.

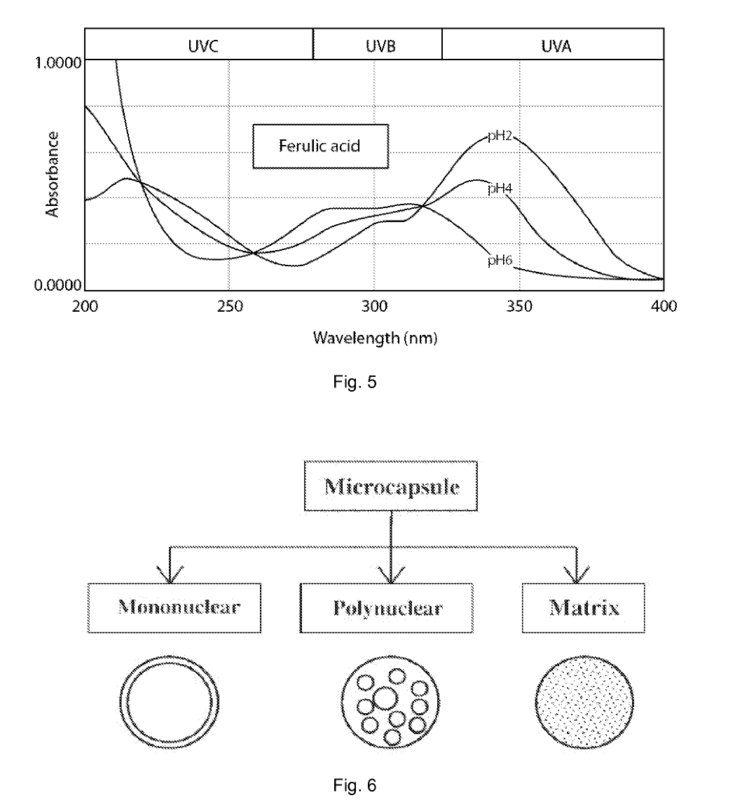

FIG. 5 shows a graphical representation of absorbance spectra (wave lengths from 200 to 400 nm; absorbance in arbitrary units) of ferulic acid at different pH values (pH 2, pH 4, pH 6) in ethanol/water. The pH was adjusted with NaOH/HCl in aqueous solution.

FIG. 6 shows schematical representations of different microcapsule morphologies (mononuclear, polynuclear, matrix).

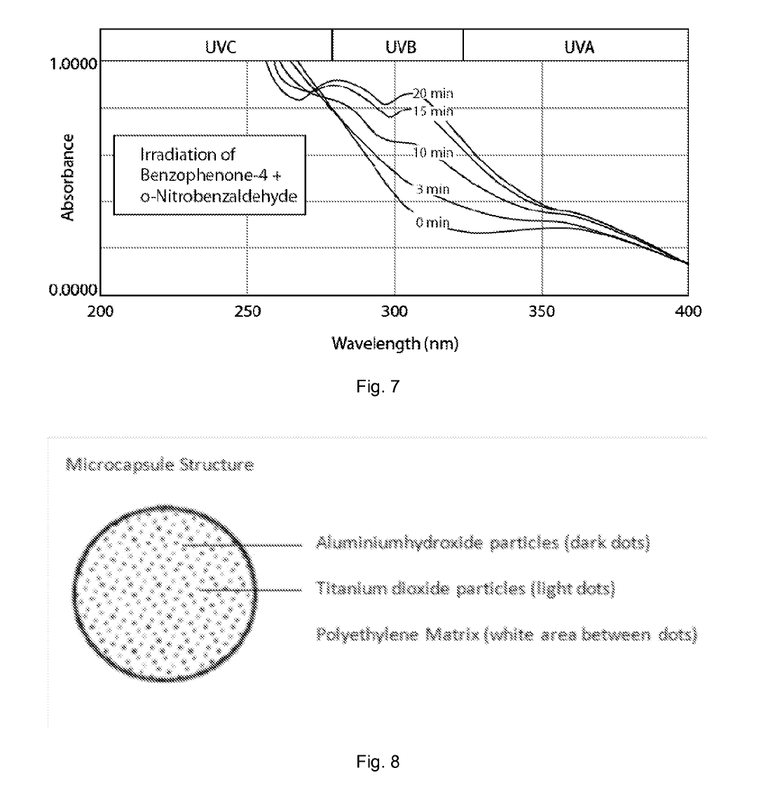

FIG. 7 shows a graphical representation of absorbance spectra (wave lengths from 200 to 400 nm; absorbance in arbitrary units) of a mixture of the sunscreen agent benzophenone-4 and o-nitrobenzaldehyde (photoacid generator) at pH 10 in acetonitrile/water (1:1) taken at the indicated time points after admixing (and hence exposure to UV radiation) the components.

FIG. 8 shows a schematic representation of the microcapsule structure of aluminium hydroxide particles (dark dots) and titanium dioxide particles (light dots) in a polyethylene matrix (white area between the dark and light, respectively, dots).

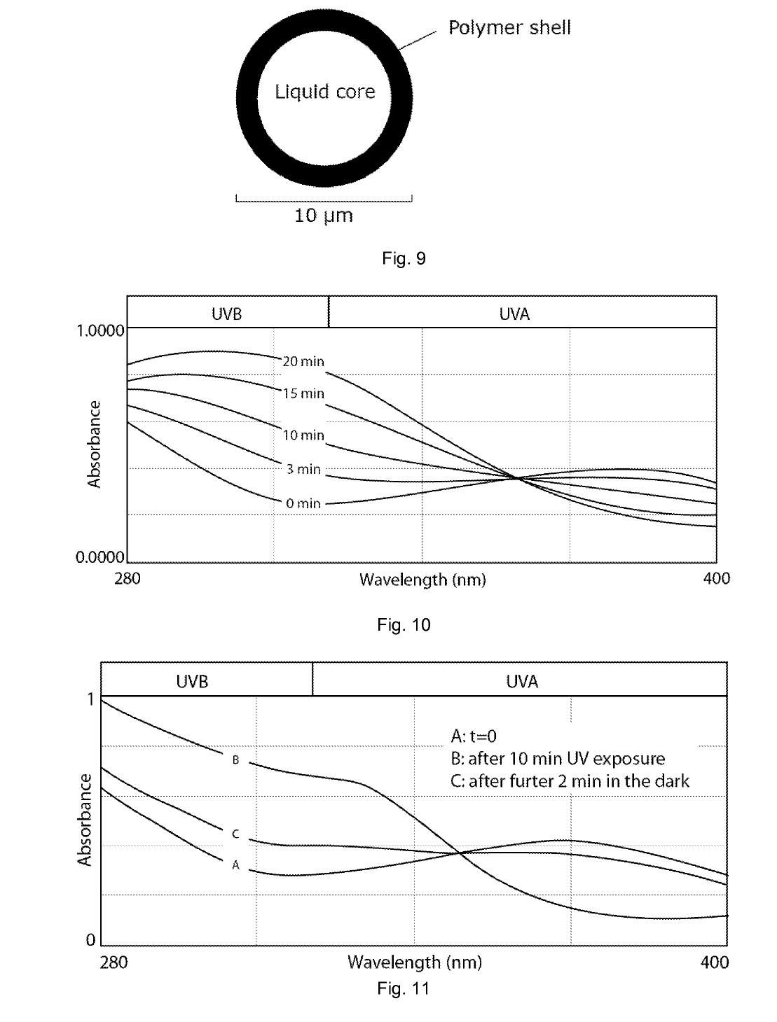

FIG. 9 shows a schematic representation of one particular microcapsule morphology useful for certain embodiments of the invention. A polymer shell encloses a liquid core that may contain various components in an aqueous- or organic-based solvent.

FIG. 10 shows a graphical representation of predicted absorbance spectra (wave lengths from 280 to 400 nm, absorbance in arbitrary units) of a sunscreen formulation of Example 3 (comprising microcapsules having a polymer shell and a liquid core that includes deprotonated (inactivated) benzophenone-4 sunscreen agent and 3-nitrobenzaldehyde, an irreversible photoacid progenitor compound) for the indicated time points after exposure to UV irradiation.

FIG. 11 shows a graphical representation of predicted absorbance spectra (wave lengths from 280 to 400 nm, absorbance in arbitrary units) of a sunscreen formulation of Example 4 (comprising microcapsules having a polymer shell and a liquid core that includes deprotonated (inactivated) benzophenone-4 sunscreen agent and 1-(2-nitroethyl)-2-naphthol, a reversible photoacid progenitor compound). Curve A: before exposure to UV irradiation; curve B: at the indicated time point after exposure to UV irradiation; and curve C: after removing UV irradiation and keeping the sunscreen formulation in the dark as indicated.

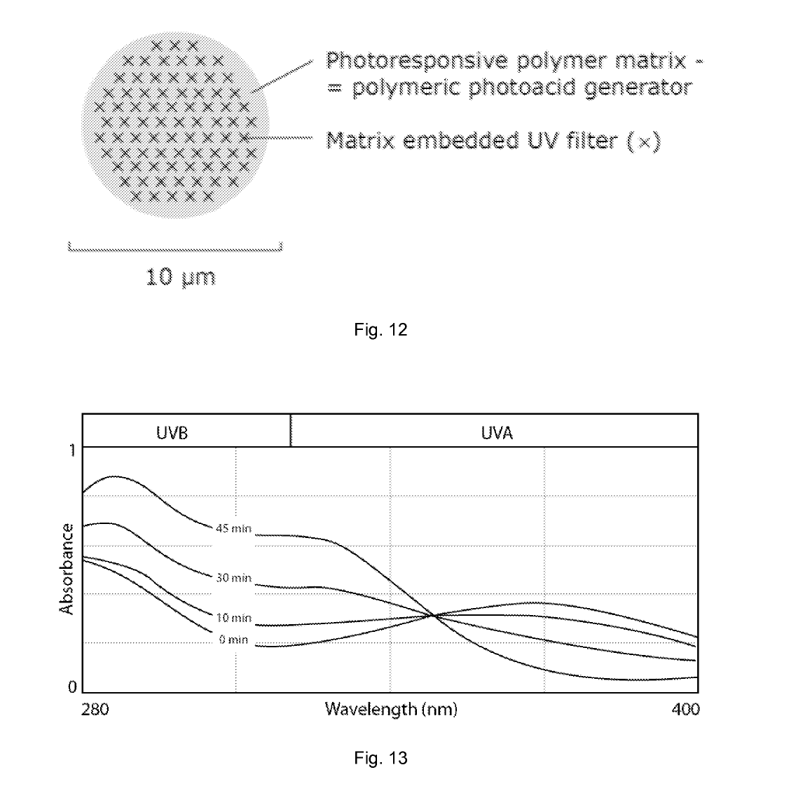

FIG. 12 shows a schematic representation of another particular microcapsule morphology useful for other certain embodiments of the invention. A photoresponsive polymer matrix (in this case a polymer that is a photoacid generator) has one or more components embedded within it, including a sunscreen agent (also known as a UV filter) which may be inactivated by protonation or deprotonation.

FIG. 13 shows a graphical representation of predicted absorbance spectra (wave lengths from 280 to 400 nm, absorbance in arbitrary units) of a sunscreen formulation of Example 5 (comprising microcapsules having a photoresponsive polymer matrix that upon exposure to UV radiation produces protons that are capable of protonating the deprotonated (inactivated) benzophenone-4 sunscreen agent that is embedded within the matrix) for different time points after exposure to UV radiation as indicated.

The following non-limiting examples further illustrate the present invention:

### EXAMPLES

### Example 1

### Photoreactive Composition

Mix equimolar amounts of o-nitrobenzaldehyde (10 mM), a photoacid progenitor compound, and benzophenone-4 (10 mM), a sunscreen agent, in water/acetonitrile (1:1). Adjust to pH 10 with NaOH, causing the sunscreen agent to take a deprotonated form that absorbs less UV radiation than the protonated form. Irradiate with Phillips Sun Simulator (Type HB 175/A) (produces UV-containing light similar to natural sunlight) positioned at a distance of 27.5 cm from the sample, producing a UV-intensity of UV-Index 5, equivalent to midday July sunlight in Zurich, Switzerland (8.54° E 47.38° N). The UV Index is measured using a standard UV intensity meter (Oregon Scientific UV888). Take samples and measure absorbance in Spectrometer Beckmann DU-640 at time 0 and after 3, 10, 15 and 20 min.

Assay Method:

An aliquot of sample is placed between two quartz glass plates of a Beckmann DU-640 Spectrometer to form a layer of ca. 0.1 mm thickness. UV absorbance spectra is recorded before UV irradiation (t=0) and after various periods of UV irradiation, using the Phillips Sun Simulator described above.

FIG. 7 shows the results of the absorbance (arbitrary units) measurements demonstrating that the UV absorbance, particularly in the UVB region, is increasing over time due to protonation of benzophenone-4 by o-nitrobenzaldehyde (cf. also the pH-dependent absorbance of benzophenone-4 shown in FIG. 1).

### Example 2

### Photobase Generating System, and Photoresponsive Sunscreen Formulation Using Same

Nanocomposite microcapsules (matrix structure, cf. FIG. 6 and FIG. 8) of an average size of 0.1 mm diameter containing a mixture of solid aluminium hydroxide particles (diameter ca. 0.1 μm) and solid titanium dioxide particles (diameter ca. 0.1 μm) in a polymer matrix of polyethylene form a photobase generating system: photolabile polyethylene degrades upon UV radiation which leads to the release of aluminium hydroxide thus lowering the pH of the medium containing the microcapsules.

Preparation: 50 g of polyethylene is heated to 150° C. until molten. 25 g of TiO₂ powder (average particle size 25 nm) and 25 g of fine powdered aluminum hydroxide are added. The composition is slowly stirred treated with hourly ultrasonic pulses for 1 day at 150° C. under inert atmosphere. The mixture is poured drop wise into ice cold water. Solid particles are grinded in an electrical mortar to obtain a fine powder. The powder is then extensively washed with dilute HCl.

A cosmetic balm is prepared as a cosmetically acceptable carrier. Briefly, the following ingredients are heated to 80° C. and stirred to obtain a homogeneous clear transparent liquid (all figures % w/w): 49% Castor (Ricinus cornmunix) oil; 13% octyldodecanol; 8% beeswax (cera alba); 6% micronized titanium dioxide; 5% ozokerite; 8% Candililla (Euphorbia cerifera) wax; 5% myristyl lactate; and 6% petrolatum. To this cosmetically acceptable carrier is added 10% (w/w) of the nanocomposite microcapsules prepared as above and 7% (w/w) of inactivated (by protonation) p-aminobenzoic acid (PABA) (prepared as described below). The resulting mixture is homogenised for 20 seconds with a rotor-stator homogenizer (Ultraturrax, T25, IKA-Labortechnik) at 1000 rpm and quickly cooled to 20° C. in a water bath to form a photoresponsive sunscreen formulation.

Samples of this sunscreen formulation are tested for photoresponsive activity. Measurements of in-vitro SPF at time point 0 and different time points after exposure to UV radiation indicate an increase of SPF upon UV irradiation. SPF increases over time as this sunscreen formulation is exposed to UV radiation. UV irradiation causes release of base (hydroxide ions) from the photobase generating system, hence causing deprotonation of the inactivated (protonated) form of sunscreen agent to form the activated form of the sunscreen agent.

In Vitro SPF Assay:

Sample is evenly spread over the surface of a roughened PMMA plate (Helioplates from Helioscience, Marseille, France, 16 cm2, specific roughness: Ra=6-7 Im) at 1.2 mg/cm² and is allowed to settle for 20 min. After irradiation with a Phillips Sun Simulator (Type HB 175/A) lamp for various periods of time the in vitro SPF is measured using an Optronic OL 754 Spectroradiometer containing an integrating sphere behind the sample and using a double monochromator/photo multiplier tube (PMT) detection system.

Inactivation of PABA:

PABA, a sunscreen agent inactivated by protonation, is prepared as follows. A 1 litre round-bottomed flask equipped with a paddle agitator, and nitrogen gas inlet is immersed in a water bath and kept at 10° C. with a thermostat. Acetone (500 mL) and PABA [Fluke; 06930]; (50 g) is added to this flask, and the mixture stirred under nitrogen for 20 min at 20° C. to remove oxygen. After complete solubilisation of the sunscreen agent an equimolar amount of conc. HCl is added to protonate the sunscreen agent. The inactivation of the sunscreen by protonation is checked by monitoring the change in UV absorbance using a Beckman DU 610 photospectrometer. The solution is rotary evaporated and dried under vacuum to prepare a solid form of protonated PABA, a sunscreen agent inactivated by protonation. This resulting sample of aminophenone sunscreen agent (PABA) is substantially in its protonated form and hence absorbs less UV radiation in such an inactive form (see FIG. 3).

### Example 3

### Irreversible Photoacid Generating System, and Microcapsulated Photoresponsive Sunscreen Formulation Using Same

A sunscreen formulation comprising microcapsules of one embodiment of the invention (See FIG. 9) contains microcapsules which comprise an aqueous liquid core containing: (1) an inactivated (deprotonated) sunscreen agent; and (2) an irreversible photoacid progenitor compound. The sunscreen formulation comprising the microcapsules of this example is formed by suspending 1 g of the microcapsules in 10 ml of sunflower oil, and samples of this formulation are tested for photoresponsive activity. FIG. 10 shows predicted results of absorbance measurements (arbitrary units, which may be collected using the assay method described in Example 1). UV absorbance in the UVB region is predicted to increase over time as this sunscreen formulation is exposed to UV irradiation.

Initially, the sunscreen agent is in its deprotonated (inactive) form. Exposure to UV irradiation generates acid from the photoacid progenitor compound and this causes a drop in pH which protonates the (previously deprotonated) sunscreen agent. Upon protonation, the sunscreen agent thus switches and reverts to its high UV absorbing state.

The microcapsules for this example are prepared as follows. In an aqueous-phase mixture of acetone (20 g) and water (1 g) is dissolved: poly(methylmethacrylate) (PMMA) (1 g), 3-nitrobenzaldehyde (1.25 g) [SigmaAldrich; 72780] (an irreversible photoacid), and benzophenone-4 sodium salt (1 g) (an inactivated sunscreen agent). Sorbitan monoolerate (Span 80, Fluke; 85548) (2 g) is dissolved in white mineral oil (103 g), this is placed in a 200 mL heat-jacketed glass vessel kept at 20° C. by a thermostat, and this oil solution is sheered using a rotor-stator homogenizer (Ultraturrax, T25, IKA-Labortechnik) at 1500 rpm. To this mixing oil-phase is slowly added (over a 60 sec period) the aqueous-phase prepared above and emulsified by keeping the homogenizer speed at 1500 rpm for approximately 30 min to form an acetone/oil emulsion. The polymer shell is precipitated by removal of the acetone using a rotary evaporator to form the microcapsules.

Inactivation of benzophenone-4:

Benzophenone-4 sodium salt, a sunscreen agent inactivated by deprotonation, is prepared as follows. A 1 litre round-bottomed flask equipped with a paddle agitator, and nitrogen gas inlet is immersed in a water bath kept at 10° C. with a thermostat. Ethanol (500 mL) and benzophenone-4 [BASF/Uvinul MS 40]; (50 g) is added to this flask, and the mixture stirred under nitrogen for 20 min at 20° C. to remove oxygen. After complete solubilisation of the sunscreen agent an equimolar amount of NaOH is added to deprotonate the sunscreen agent. The inactivation of the sunscreen by deprotonation is checked by monitoring the change in UV absorbance using a Beckman DU 610 photospectrometer. The solution is rotary evaporated and dried under vacuum to prepare a solid form of benzonphenone-4 sodium salt, a sunscreen agent inactivated by deprotonation.

Poly(methylmethacrylate) (PMMA), W=15000, can be obtained from a variety of suppliers (for example, SigmaAldrich) or can be synthesised according to well-known procedures such as free-radical solution polymerisation as follows. A 1 litre, round-bottomed flask equipped with a paddle agitator, reflux condenser, and nitrogen gas inlet is immersed in a thermostat-regulated oil bath and kept at 60.1° C. Tetrahydrofuran (THF) (538 g) is charged to the flask, methylmethacrylate (MMA) (178.15 g) is added, and the mixture stirred under nitrogen for 20 min at 70° C. to remove oxygen. Azobisisobutyronitrile (AIBN) (0.90 g in 2.0 g THF) as a radical starter is then added to initiate polymerisation and the polymerisation reaction allowed to continue at 70° C. for 21 h. The polymer is then precipitated by slowly pouring the solution into a fourfold excess of ethanol with stirring, dried to constant mass in a vacuum oven at 40° C. (20 h) and ground in an electrical mortar to obtain a fine powder.

### Example 4

### Reversible Photoacid Generating System, and Microcapsulated Photoresponsive Sunscreen Formulation Using Same

A sunscreen formulation according to another embodiment of the invention (See FIG. 9) contains microcapsules which comprise an aqueous liquid core containing: (1) an inactivated (deprotonated) sunscreen agent; and (2) a reversible photoacid progenitor compound. The sunscreen formulation comprising the microcapsules of this example is analogously formed and can be tested for reversible photoresponsive activity as described in Example 3, using optimal UV irradiation. FIG. 11 shows predicted absorbance values (arbitrary units, according to the assay method described in Example 1). UV absorbance in the UVB region increases over time as this sunscreen formulation is exposed to UV irradiation, and then that UV absorbance in the UVB region decreases after ceasing exposure to UV irradiation and keeping the composition in the dark.

Initially, the sunscreen agent is in its deprotonated (inactive) form. Exposure to UV irradiation reversibly generates acid from the photoacid progenitor compound and this causes a pH drop which protonates the (previously deprotonated) sunscreen agent. Upon protonation, the sunscreen agent thus switches and reverts to its high UV absorbing state. Upon removal of UV irradiation, the photoacid compound absorbs the free acid, hence raising the pH in the core of the microcapsule and hence causing deactivation (by deprotonation) of the sunscreen agent.

The microcapsules for this example can be prepared as described in Example 3 except that 1-(2-nitroethyl)-2-naphthol (1.3 g) (a reversible photoacid), is used in place of 3-nitrobenzaldehyde. The reversible photoacid progenitor compound (1-(2-nitroethyl)-2-naphthol) is synthesized as described in “Nunes et al, Photoacid for Extremely Long-Lived and Reversible pH-Jumps, J. AM. CHEM. SOC. 2009, 131, 9456-9462”

### Example 5

### Irreversible Photoacid Generating System Using a Polymeric Photoacid, and Microcapsulated Photoresponsive Sunscreen Formulation Using Same

A sunscreen formulation according to another embodiment of the invention (See FIG. 12) contains matrix-based microcapsules which comprise: (1) an inactivated (deprotonated) sunscreen agent; and (2) an irreversible photoacid progenitor polymer. The sunscreen formulation comprising the microcapsules of this example is formed by dispersing 1 g of such microcapsules in 10 mL of an oil-in-water (O/W) emulsion (see below) and samples of this formulation are tested for photoresponsive activity. FIG. 13 shows predicted absorbance values (arbitrary units, according to the assay method described in Example 1 except by exposing to UV irradiation at a UV index of 6, by positioning the Sun Simulator at a distance of 31 cm from the sample). The UV absorbance in the UVB region increases over time as this sunscreen formulation is exposed to UV irradiation.

Upon exposure to UV radiation, the photoacid progenitor polymer, comprising poly(vinylchloride) (PVC) photosensitised by the presence of hydroquininone in the matrix of the microcapsule, generates HCl which then protonates the inactivated sunscreen agent (initially in a deprotonated form). Upon protonation, the sunscreen agent thus switches and reverts to its high UV absorbing state.

The microcapsules for this example can be prepared as follows. High molecular weight PVC polymer (50 g) [SigmaAldrich/81387] and hydroquinone (2 g) [SigmaAldrich/H9003] are dissolved in THF (1000 mL), and 1 g of benzophenone-4 sodium salt (made according to Example 3), an inactivated (by deprotonation) sunscreen agent, is finely dispersed within this polymer solution using a rotor-stator homogenizer (Ultraturrax, T25, IKA-Labortechnik).

Finally, to further reduce the particle size, the coarse suspension is homogenized using an ultrasonic processor (Sonic Vibracell VC750, 720 W, 20 kHz) equipped with a 13 mm tip high-intensity horn. The solution is spray-dried using a laboratory scale spray-drier (Mini Spray Dryer B-290 ADVANCED, Büchi AG, Switzerland) with an aspirator rate of 40 m²/min, a feed rate of 10 ml/min, an inlet temperature of 90° C., outlet temperature of 70° C., and a spray flow of 300 L/h.

Preparation of O/W emulsion, pH 6.5:

100 mL of an O/W emulsion is prepared as a cosmetically acceptable carrier (all figures % w/w in the final emulsion). Briefly, the water phase is prepared from: 80.6% water (aqua), deionised; 5% disodium citrate; 4% glycerine, 0.4% acrylates/C10-30 alkyl acrylate crosspolymer; and 0.2% xanthan gum. The oil phase is prepared from: 7.5% cetearyl isononanoate; 1% Euxyl PE 9010; and 0.1% parfum. The water phase is mixed with a rotor-stator homogenizer (Ultraturrax, T25, IKA-Labortechnik) at 1500 rpm and the oil phase is added over a time period of 60 seconds while stirring. The stirrer speed is kept for 15 min. The pH of the emulsion is adjusted with approximately 1.2 mL NaOH solution (10% w/w) to pH 6.5.

### Example 6

### Irreversible Photoresponsive Base Generating System Using a Base/Protonated Sunscreen Separation-Based Approach, and Photoresponsive Sunscreen Formulation Using Same

A sunscreen formulation comprising microcapsules of another embodiment of the invention (See FIG. 9) contains microcapsules which comprise an aqueous liquid core containing: (1) an inactivated (protonated) sunscreen agent; and (2) a UV decomposable and gas generating progenitor compound. The sunscreen formulation comprising the microcapsules of this example is formed by dispersing 2 g of the microcapsules in 10 ml of an oil-in-water (O/W) emulsion (see below). This emulsion is buffered to pH 6.5 (a basic pH relative to the protonated sunscreen agent) by a citrate buffer, and samples of the resulting sunscreen formulation are tested for photoresponsive activity. Measurements of UV absorbance (and/or SPF) at time point 0 and different time points after exposure to UV radiation indicate an increase of UV absorbance (and/or) SPF upon UV irradiation.

The polymer shell isolates the protonated (inactive) form of the sunscreen agent from a buffered external environment. Upon exposure to UV irradiation, a UV-decomposable and gas-generating progenitor compound, dibenzoylperoxide (DBP), also comprised within the microcapsule, generates CO₂, causes a build-up of internal pressure that then ruptures the microcapsule. This photoresponsive system then generates a situation where the buffered external environment comes into contact with the inactivated (protonated) sunscreen agent, enabling the establishment of acid/base equilibrium between the sunscreen agent, which was previously within the core of the microcapsule, and the buffered external environment. The (previously inactivated by protonation) sunscreen agent thereby becomes deprotonated and hence active, shifting and reverting the sunscreen agent to its high UV absorbance state.

Preparation of O/W emulsion, pH 6.5:

100 mL of an O/W emulsion is prepared as a cosmetically acceptable carrier (all figures % w/w in the final emulsion). Briefly, the water phase is prepared from: 59.8% water (aqua), deionised; 1% Euxyl PE 9010; 5% disodium citrate; and 3% PEG-30 dipolyhydroxystearate. The oil phase is prepared from: 15% cyclomethicone (pentamer); 8% ethylhexyl palmitate; 5% octyldodecanol; 1% hydrogenated castor oil; and 1% polyethylene. The water phase is mixed with a rotor-stator homogenizer (Ultraturrax, T25, IKA-Labortechnik) at 1500 rpm and the oil phase is added over a time period of 60 seconds while stirring. The stirrer speed is kept for 15 min. The pH of the emulsion is adjusted with approximately 1.2 mL NaOH solution (10% w/w) to pH 6.5.

In a related experiment, a second sunscreen formulation comprising such microcapsules is prepared as above but further comprises 7% of microfine titanium dioxide and 7% of microfine zinc oxide as additional sunscreen agents. This second sunscreen formulation is predicted to have a higher initial SPF (ie, before exposure to UV radiation), due largely to the presence of titanium dioxide and microfine zinc oxide. Upon exposure to UV radiation, an increase in SPF over this initial SPF is indicated.

Preparation of a foundation O/W sunscreen formulation, pH 6.5:

100 mL of an O/W emulsion is prepared as a cosmetically acceptable carrier (all figures % w/w in the final emulsion). Briefly, the water phase is prepared from: 54% water (aqua), deionised; 1% Euxyl PE 9010; 5% disodium citrate; and 3% PEG-30 dipolyhydroxystearate. The oil phase is prepared from: 10% cyclomethicone (pentamer); 7% zinc oxide (microfine); 7% titanium dioxide (microfine); 5% ethylhexyl palmitate; 4% octyldodecanol; 0.8% hydrogenated castor oil; and 2% polyethylene. The water phase is mixed with a rotor-stator homogenizer (Ultraturrax, T25, IKA-Labortechnik) at 1500 rpm and the oil phase is added over a time period of 60 seconds while stirring. The stirrer speed is kept for 15 min. The pH of the formulation is adjusted with about 1.2 mL NaOH solution (10% w/w) to pH 6.5.

The microcapsules for both sunscreen formulations of this example can be prepared as follows. An oil-phase is formed from PMMA (2.5 g) (obtained as described in Example 3) dissolved in dichloromethane (70.5 g), to which dibenzoylperoxide (0.1 g) [Fluke, 33581] and the sunscreen agent padimate-0 (3.8 g) [ISP, Escalol 507] are then added. An equal mass of an aqueous-phase surfactant solution (1% poly(methacrylic acid) (prepared according to standard procedures (Garcia et al. Synthesis and characterization of poly(methacrylic acid) hydrogels for metoclopramide delivery, European Polymer Journal 40 (2004) 1637-1643) is charged to a 200 mL heat-jacketed glass vessel kept at 20° C. with a thermostat. This aqueous-phase is stirred using a rotor-stator homogenizer (Ultraturrax, T25, IKA-Labortechnik) at 4,000 rpm, the oil-phase is slowly added (over a 60 sec period) to form an oil-in-water (O/W) emulsion. Agitation is maintained at this rate for 1 h before pouring the emulsion into an additional 120 mL of aqueous-phase surfactant solution. This diluted emulsion is then rotary evaporated for 20 min at 35° C., after which the vacuum is removed and the resulting dispersion maintained at 35° C. for a further 40 min. The resulting dispersion of (still permeable) microcapsules is then suspended in HCl (10M) at 20° C. for 8 min to protonate (and hence inactivate) the padimate-0 sunscreen agent within the liquid phase of the microcapsule. After this period of protonation, the dispersion of microcapsules is rotary evaporated, the resulting microcapsules cleaned by ultrafiltration using an appropriately sized Millipore filtration unit and then finally dried under vacuum for 24 h.

As will be appreciated by the person skilled in the art: (1) by varying the respective amounts of the (inactivated) sunscreen agent, the amount of the gas generating progenitor compound (in this case dibenzoylperoxide) and/or the physical parameters of the microcapsule shell (in this case PMMA), the sensitivity of this photoresponsive sunscreen formulation to UV radiation can be varied; and/or (2) by inclusion of a different or differing amounts of another sunscreen agent, the initial SPF or UV absorbance characteristics can be modified; in each case to provide the required characteristics of the sunscreen formulation.

What is claimed is:

1. A photoresponsive sunscreen composition comprising
(a) a sunscreen agent capable of undergoing protonation or deprotonation to form a protonated or deprotonated, respectively, sunscreen agent which absorbs, scatters or reflects more or less UV radiation than said sunscreen agent does before being protonated or deprotonated, respectively; and
(b) a photoresponsive acid or base generating system which is capable of protonating or deprotonating, respectively, said sunscreen agent when the composition is exposed to UV radiation.

2. The composition of claim 1 wherein the sunscreen agent is present substantially in its protonated or deprotonated form before the composition is exposed to UV radiation, and which said protonated or deprotonated, respectively, form of said sunscreen agent absorbs, scatters or reflects less UV radiation than said sunscreen agent does upon exposure of the composition to UV radiation.

3. The composition of claim 2 wherein the composition, upon exposure of the composition to UV radiation, shows an increase of about 5 to about 50, preferably about 10, 15, 20, 25, 30, 35 or 40 SPF units compared to said composition before exposure to UV radiation.

4. The composition of claim 2 wherein the protonated or deprotonated form of the sunscreen agent, before exposure of the composition to UV radiation, absorbs, scatters or reflects less UVA and/or UVB than said sunscreen agent does upon exposure to UV radiation.

5. The composition of claim 1 claims wherein the sunscreen agent is selected from the group consisting of a substituted benzophenone derivative of formula (I) 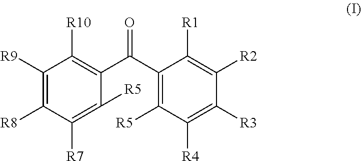
wherein
each of R₁ to R₁₀ is independently selected from the group consisting of glucopyranoxy, hydrogen, hydroxyl, nitro, cyano, amino, halide (in particular F, Cl, Br), straight or branched C₁₋₁₀-alkyl, C₁₋₁₀-alkoxy, C₂₋₁₀-alkenyl, C₂₋₁₀-alkenyloxy, C₆₋₁₀-aryl, optionally substituted with one or more groups selected from hydroxyl, C₁₋₄-alkyl, C₁₋₄-alkoxy, C₂₋₄-alkenyl or C₂₋₄-alkenyloxy, and SO₃M wherein M is hydrogen, a monovalent metal ion or a quarternary ammonium group;
an aminophenone of formula (II): 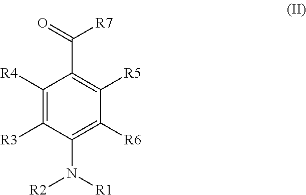
wherein
R₁ and R₂ are each independently selected from H and straight or branched C₁₋₆-alkyl or C₂₋₆-alkenyl, optionally substituted with one or more hydroxyl, R₃ to R₆ are each independently selected from H, hydroxyl, nitro, cyano, amino, halide (in particular F, Cl, Br), straight or branched C₁₋₆-alkyl or C₂₋₆-alkenyl, optionally substituted with one or more hydroxyl,
R₇ is selected from hydroxyl, straight or branched C₁₋₁₀-alkoxy, C₂₋₁₀-alkenyloxy, C₆₋₁₀-aryl, optionally substituted with one or more groups selected from hydroxyl, C₁₋₆-alkyl, C₁₋₆-alkoxy, C₂₋₆-alkenyl, C₂₋₆-alkenyloxy and C₁₋₁₀-alkoxycarbonyl, or
R₁ is —(CH₂—CH₂O)ₓH, R₂ is —(CH₂—CH₂O)_{y}H, R₇ is —O—(CH₂—CH₂O)_{z}H and each of R₃ to R₆ are H, wherein x+y+z is =25; and wherein
the substituents —NR₁R₂ and COR₇ have a para, ortho or meta orientation to one another;
a compound of formula (III): 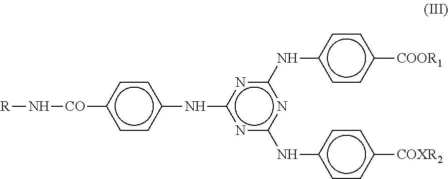
wherein:
R is a straight or branched C₁₋₈-alkyl group, C₅₋₁₂-cycloalkyl, optionally substituted with one or more straight or branched C₁₋₄-alkyl groups; X is an oxygen atom or the group —NH—; R₁ has the same meanings as R, or is hydrogen, a monovalent metal ion, a quaternary ammonium group, or a group of formula (IV) 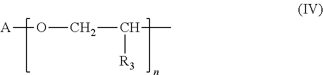
in which A is a straight or branched C₁₋₈-alkyl, C₅₋₁₂-cycloalkyl, or C₆₋₁₀-aryl optionally substituted with one or more straight or branched C₁₋₄-alkyl groups; R₃ is hydrogen or methyl, n is an integer from 1 to 10; R₂ has the same meaning as R when X is —NH—, or has the same meanings as R₁ when X is oxygen;
a benzylidenecamphor derivative of formula (V): 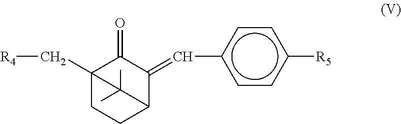
wherein R₄ is hydrogen, or the group SO₃M, in which M is hydrogen, or a monovalent metal ion, or a quaternary ammonium group, and R₅ is hydrogen, methyl, a group SO₃M wherein M is defined as above, or a group of formula VI or VII 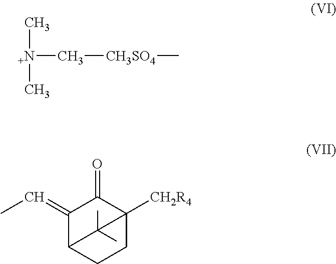
wherein R₄ is as defined above for formula (V);
a dibenzoylmethane derivative of formula (VIII): 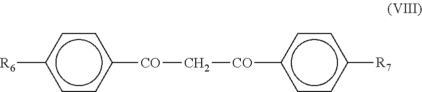
in which R₆ and R₇ are selected independently from hydrogen, straight or branched C₁₋₈-alkyl and straight or branched C₁₋₈-alkoxy;
an alkoxycinnamic acid ester of formula (IX): 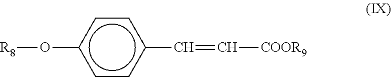
wherein R₈ is a straight or branched C₁₋₈-alkyl group, and R₉ is selected from hydrogen, a straight or branched C₁₋₁₀-alkyl group, a monovalent metal ion and a quaternary ammonium group;
a triazinoaniline derivative of formula (X): 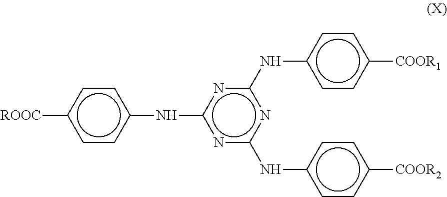
in which R, R₁ and R₂ are defined as above for formula (III);
a diphenylcyanoacrylate of formula (XI): 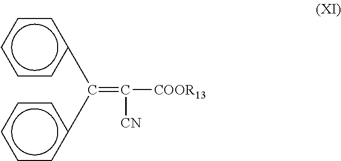
wherein R₁₃ has the same meanings as R₁ defined above for formula (III);
a salicylic acid derivative of formula (XII): 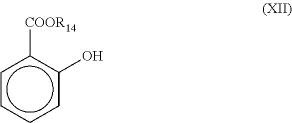
wherein R₁₄ is hydrogen, or a straight or branched C₁₋₁₀-alkyl group, a benzyl group optionally substituted with a straight or branched C₁₋₆-alkyl group, a 3,3,5-trimethylcyclohexyl residue, both as a racemate and as any optically active forms, or the group HN⁺(CH₂CH₂OH)₃;
a benzimidazolesulfonic acid derivative of formula (XIII): 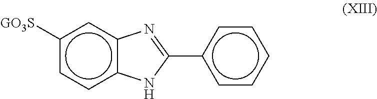
in which G is hydrogen, or a monovalent metal ion, or a quaternary ammonium group; and
a quarternary ammonium salt of a para-dialkylamino benzamide compound of formula (XIV) 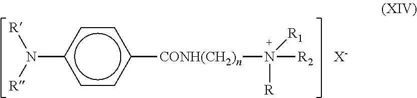
wherein R′ and R″ are each C₁₋₂-alkyl;
n is an integer of from 2 to 6;
R is a linear, branched or cyclic alkyl radical having from 1 to 30 carbon atoms;
R₁ and R₂ are each selected from hydrogen and C₁₋₄-alkyl or, alternatively, R₁ and R₂, together with the attached cationic nitrogen atom can form a 5- to 6-membered heterocyclic ring selected from the group consisting of 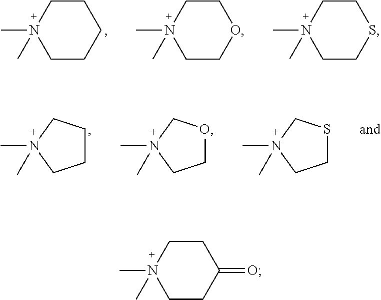
and X is an anion, preferably selected from the group consisting of chloride, bromide, sulphate, sulfonate, haloacetal and aryl sulfonates.

6. The composition of claim 5 wherein the sunscreen agent is an aminophenone selected from the group consisting of p-aminobenzoic acid (PABA), ethylhexyl dimethyl PABA (padimate-O), penthyl dimethyl PABA (padimate-A), PEG-25 PABA (lisidimate), glyceryl PABA, ethyl dihydroxypropyl PABA (roxadimate), menthyl anthranilate (meradimate) and diethylamino hydroxybenzoyl hexyl benzoate or a benzophenone selected from the group consisting of benzophenone-1, benzophenone-2, benzophenone-3, benzophenone-4, benzophenone-5, benzophenone-6, benzophenone-8 and benzophenone-9, or is camphor benzalkonium methosulfate.

7. The composition of claim 1 wherein the photoresponsive acid or base generating system comprises a photoacid or photobase progenitor compound.

8. The composition of claim 7 wherein the photoacid progenitor compound is selected from the group consisting of onium salts, 2-nitrobenzyl esters of sulfonic acids, 2 nitrobenzyl esters of carboxylic acids, imino sulfonate, 1-oxo-2-diazonaphthoquinone-4-sulfonate derivatives, 1-oxo-2-diazonaphthoqiunone-5-arylsulfonate derivatives, N-hydroxyimide sulfonate, tri(methanesulfonyloxy)-benzene, triarylphosphate derivatives, azophenol derivatives, naphthol derivatives, 4-phenoxyphenyl)diphenylsulfonium triflate, nitro-substituted aromatic aldehydes, 2-hydroxyphenyl-1-(2-nitrophenyl)ethyl phosphate, 1-(2-nitrophenyl)ethyl sulfate and spirooxazine photochromes.

9. The composition of claim 7 wherein the photobase progenitor compound is selected from the group consisting of carbamates, O-acyloximes, ammonium salts, sulfonamides, formamides, nifedipines, α-aminoketones, acridine, 6-methoxyquinoline, styrene, 2-vinylnaphthalene, 2-naphthylacetylene, nitro-substituted phenylacetylenes, nitro-substituted phenylalkenes, p-nitrophenylacetylene, hydroxyl-substituted vinylnaphthalenes, hydroxyl-substituted naphthylacetylenes, o-hydroxystyrene, methoxy-substituted benzyl alcohols, dimethoxy-substituted benzylalcohols, triphenylmethane leucohydroxide derivatives, 9-phenylxanthen-9-ol, trans-retinol, dibenzosuberol, 5-suberol, diarylmethanol, triarylmethanol, pyridoxine and 9-hydroxy fluorine.

10. The composition of claim 1 wherein the photoresponsive acid or base generating system comprises
(i) an acid or base, respectively, present in microcapsules which become at least partially permeable for said acid or base, respectively, or for said sunscreen agent upon exposure to UV radiation; or
(ii) an acid or a base, respectively, and the sunscreen agent being present in microcapsules which become at least partially permeable for said acid or base, respectively, or for said sunscreen agent upon exposure to UV radiation.

11. The composition of claim 10 wherein, upon UV radiation, said microcapsules become permeable for hydrogen ions and/or hydroxide ions and/or rupture.

12. The composition of claim 11 wherein the microcapsules rupture by an increase of internal gaseous pressure.

13. The composition of claim 10 wherein said acid or base, respectively, is a second sunscreen agent which protonates or deprotonates, respectively, the (first) sunscreen agent when said microcapsules become at least partially permeable for said first or second sunscreen agent, preferably wherein said first sunscreen agent is an aminophenone and said second sunscreen agent is a benzophenone.

14. The composition of claim 1 wherein the photoresponsive acid or base generating system is a reversible system.

15. The composition of claim 1 further comprising:
at least one further organic sunscreen agent, preferably one selected from the group consisting of 3-benzylidene camphor, benzylidene camphor sulfonic acid, β-2-glucopyranoxy propyl hydroxyl benzophenone, bis-ethylhexyloxyphenol methoxyphenyl triazine, butyl methoxydeibenzoylmethane, camphor benzalkonium methosulfate, cinoxyte, DEA methoxycinnamate, diethylhexyl butamido trazone, digalloyl trioleate, diisopropyl methyl cinnamate, dimethoxyphenyl-[I-(3,4)J-4,4-dimethyl 3-pentanedione, disodium phenyl dibenzylimidazole tetrasulfonate, drometrizole, drometrizole trisiloxane, ethylhexyl dimethoxy benzylidene dioxoimidazoline propionate, ethylhexyl methoxycinnamate, ethylhexyl salicylate, ethylhexyl triazone, ferulic acid, glycerylethylhexanoate dimethoxycinnamate, homosalate, isoamyl p-methoxycinnamate, isopenfyl trimethoxycinnamate trisiloxane, isopropylbezyl salicylate, isopropyl methoxycinnamate, menthyl anthranilate, 4-menthylbenzylidene camphor, methylene bis-benzotriazolyl tetramethylbutylphenol, octocrylene, phenylbenzimidazole sulfonic acid, polyacrylamido methylbenzylidene camphor, polysiliconce-15, salicylic acid, TEA salicylate and terephthalylidene dicamphor sulfonic acid; and/or at least one inorganic sunscreen agent, preferably one selected from the group consisting of metal oxides having an atomic number ranging from 10 to 40.

16. A microcapsule comprising a sunscreen agent capable of undergoing protonation or deprotonation to form a protonated or deprotonated, respectively, sunscreen agent which absorbs, scatters or reflects more or less UV radiation than said sunscreen agent does before being protonated or deprotonated, respectively, wherein said sunscreen agent is present in said microcapsule substantially in its protonated or deprotonated form that absorbs, scatters or reflects less UV radiation than said sunscreen agent does upon protonation or deprotonation, respectively.

17. The microcapsule of claim 16 wherein the sunscreen agent is present substantially in its protonated form before the microcapsule is exposed to UV radiation.

18. The microcapsule of claim 16 wherein the sunscreen agent is defined as in claim 5 or 6.

19. The microcapsule of claim 16 wherein the microcapsule:
becomes at least partially permeable for said sunscreen agent or for an acid or a base, respectively, upon exposure to UV radiation; or
further comprises a photoacid or photobase progenitor compound.

20. A cosmetic sunscreen formulation comprising the composition claim 1 or microcapsules of claim 19 in combination with a cosmetically acceptable carrier.

21. A dermatological formulation comprising the composition of claim 1 or microcapsules of claim 19 in combination with a dermatologically acceptable carrier.

22. (canceled)

23. A method for the preparation of the composition of claim 1 comprising the step of combining components (a) and (b).

24. (canceled)

25. (canceled)

26. (canceled)

27. A cosmetic method for protecting skin and/or hair against UV radiation comprising the step of applying an effective amount of the cosmetic sunscreen formulation of claim 20 onto skin and/or hair.

28. A method for modifying the SPF of the cosmetic sunscreen formulation of claim 20 comprising the step of exposing said formulation to an effective amount of UV radiation.

29. (canceled)

30. A method for modifying the SPF of the dermatological formulation of claim 21 comprising the step of exposing said formulation to an effective amount of UV radiation.

31. A method for the prevention of skin cancer or sunburn comprising the application of an effective amount of the dermatological formulation of claim 21 onto human or animal skin.
